(19) United States
(12) Patent Application Publication
(10) Pub. No.: US 2026/0248792 A1
(43) Pub. Date: Aug. 27, 2026

(54) CANCER TREATMENT USING TOPOISOMERASE I INHIBITORS AND PLK1 INHIBITORS

(71) Applicant: CARDIFF ONCOLOGY, INC., San Diego, CA (US)

(72) Inventors: Maya RIDINGER, San Diego, CA (US); Errin SAMUELSZ, San Diego, CA (US); Tod SMEAL, San Diego, CA (US)

(21) Appl. No.: 18/870,681

(22) PCT Filed: May 30, 2023
(86) PCT No.: PCT/US2023/067641

§ 371 Date: Dec. 1, 2024

Related U.S. Application Data
(60) Provisional application No. 63/347,505, filed on May 31, 2022.
(60) Provisional application No. 63/404,824, filed on Sep. 8, 2022.

(51) Int. Cl. A61K 31/519; A61K 31/404; A61K 31/4439; A61K 31/4709; A61K 31/4745; A61K 31/5025; A61K 31/506; A61K 31/513; A61K 31/517; A61K 38/17; A61K 47/68; A61P 35/00; C07K 16/22; C07K 16/28

(52) U.S. Cl. CPC A61K 31/519; A61K 31/404; A61K 31/4439; A61K 31/4709; A61K 31/4745; A61K 31/5025; A61K 31/506; A61K 31/513; A61K 31/517; A61K 38/179; A61K 47/6811; A61P 35/00; C07K 16/22; C07K 16/2863

(57) ABSTRACT

Disclosed herein include methods, compositions, and kits suitable for use in treating cancer. In some embodiments, the method comprises administrating a topoisomerase I inhibitor and onvansertib to a subject with cancer, thereby inhibiting progression of the cancer. The method can further comprise an angiogenesis inhibitor. The method can include sensitizing cancer cells to a topoisomerase I inhibitor, by contacting the cancer cells with a composition comprising onvansertib.

### RELATED APPLICATIONS

The present application is a U.S. national phase application under 35 U.S.C. § 371 of International Application No. PCT/US2023/067641, filed on May 30, 2023 and published as WO 2023/235716 A2 on Dec. 7, 2023, which claims the benefit under 35 U.S.C. § 119(e) of U.S. Provisional Patent Application Ser. No. 63/347,505, filed on May 31, 2022; and U.S. Provisional Patent Application Ser. No. 63/404,824, filed on Sep. 8, 2022. The contents of these related applications are incorporated herein by reference in their entirety for all purposes.

### BACKGROUND

### Field

The present disclosure relates generally to the field of treatment for cancer. More specifically, combination therapies for treating cancer using topoisomerase I inhibitors in combination with polo-like kinase 1 (PLK1) inhibitors are provided. Combination therapies for treating cancer using topoisomerase I inhibitors and/or angiogenesis inhibitors in combination with polo-like kinase 1 (PLK1) inhibitors are also provided.

### Description of the Related Art

Chemotherapy based on fluoropyrimidines and oxaliplatin (FOLFOX) or irinotecan (FOLFIRI) remains first- and second-line standard-of-care for RAS-mutated metastatic colorectal cancer (mCRC) patients. Resistance to chemotherapy limits the effectiveness of treatment and results from either intrinsic resistance, or development of resistance during treatment. Overcoming resistance has the potential of increasing treatment benefit and represents a highly unmet medical need for cancer patients, including patients with RAS mutations and patients resistant to irinotecan treatment.

### SUMMARY

Disclosed herein include methods for treating cancer. In some embodiments, the method comprises: administrating a topoisomerase I inhibitor and onvansertib to a subject with cancer, thereby inhibiting progression of the cancer. In some embodiments, the subject has received a topoisomerase I inhibitor therapy and/or is known to be resistant to the topoisomerase I inhibitor therapy.

The cancer can be, for example, colorectal cancer, head and neck cancer, lung cancer, intrahepatic cholangiocarcinoma (iCCA), gastric cancer, urothelial cancer, endometrial cancer, cervical cancer, rhabdomyosarcoma, cholangiocarcinoma, glioblastoma, low-grade glioma, ovarian cancer, prostate adenocarcinoma, thyroid carcinoma, endometrial cancer, gallbladder cancer, breast cancer, or a combination thereof. In some embodiments, the cancer is colorectal cancer. In some embodiments, the colorectal cancer is selected from colorectal adenoma, colon cancer, rectal cancer, colorectal carcinoma, colorectal adenocarcinoma, gastrointestinal carcinoid tumors, gastrointestinal stromal tumors (GISTs), primary colorectal lymphomas, colon leiomyosarcomas, rectal leiomyosarcomas, colon melanomas, rectal melanomas, colorectal squamous cell carcinoma, and a combination thereof. In some embodiments, the cancer is metastatic colorectal cancer (mCRC).

In some embodiments, the mCRC is associated with a RAS mutation e.g., the RAS mutation is an HRAS mutation, a KRAS mutation and/or an NRAS mutation. In some embodiments, the RAS mutation is a KRAS mutation and/or an NRAS mutation. In some embodiments, the RAS mutation is in codon 12, codon 13, codon 61 or a combination thereof. In some embodiments, the RAS mutation is selected from KRASG12C, KRASG12D, KRASG12V, KRASG13C, KRASG13D, KRASQ61L, KRASQ61H, KRASQ61R, NRASQ61R, NRASQ61K, NRASQ61L, NRASQ61H, HRASG12V, HRASQ61R, HRASG12S and a combination thereof. In some embodiments, the RAS mutation is NRASQ61R, KRASG12C, KRASG12D, or KRASG12V.

The method can further comprise administrating an angiogenesis inhibitor to the subject. The angiogenesis inhibitor can be, for example, aflibercept, axitinib, bevacizumab, cabozantinib, lenvatinib, pazopanib, ponatinib, ramucirumab, ranibizumab, regorafenib, sorafenib, sunitinib, vandetanib, aflibercept, cetuximab, panitumumab, and a combination thereof. In some embodiments, the angiogenesis inhibitor is bevacizumab.

The method can further comprise administrating to the subject one or more additional therapeutic agents. In some embodiments, the one or more additional therapeutic agents comprise leucovorin, fluorouracil (5-FU) or a combination thereof.

In some embodiments, onvansertib and the topoisomerase I inhibitor are co-administered simultaneously. In some embodiments, onvansertib and the topoisomerase I inhibitor are administered sequentially. In some embodiments, the angiogenesis inhibitor and/or the one or more additional therapeutic agents are co-administered simultaneously with onvansertib and/or the topoisomerase I inhibitor. The angiogenesis inhibitor and/or the one or more additional therapeutic agents can be administered sequentially with onvansertib and/or the topoisomerase I inhibitor. In some embodiments, onvansertib and the topoisomerase I inhibitor are administered through different routes or through the same route. In some embodiments, the angiogenesis inhibitor and/or the one or more additional therapeutic agents are administered through different routes from onvansertib and/or the topoisomerase I inhibitor. In some embodiments, the angiogenesis inhibitor and/or the one or more additional therapeutic agents are administered through the same route as onvansertib and/or the topoisomerase I inhibitor. In some embodiments, one or more of onvansertib, the topoisomerase I inhibitor, the angiogenesis inhibitor, and the one or more additional therapeutic agents are administered orally, intraperitoneally, intravenously, subcutaneously, intramuscularly, intrathecally, or by inhalation.

In some embodiments, one or more of onvansertib, the topoisomerase I inhibitor, the angiogenesis inhibitor, and the additional one or more additional therapeutic agents are administered in a cycle of at least about 7 days, a cycle of at least about 14 days, a cycle of at least about 21 days, or a cycle of at least about 28 days. In some embodiments, onvansertib, the topoisomerase I inhibitor, the angiogenesis inhibitor, and/or the one or more additional therapeutic agents are administered in a cycle of about 28 days. In some embodiments, onvansertib is administered on at least four days in the cycle. In some embodiments, the cycle is of 28 days and onvansertib is administered on days 1-5 and 15-19 in the cycle. In some embodiments, the topoisomerase I inhibitor, the angiogenesis inhibitor, and/or the one or more additional therapeutic agents are administered on at least one day in the cycle. In some embodiments, the cycle is of 28 days and the topoisomerase I inhibitor, the angiogenesis inhibitor, and/or the one or more additional therapeutic agents are administered on days 1 and 15 in the cycle. In some embodiments, the subject undergoes at least two cycles of administration.

Onvansertib can be administered at 8 mg/m²-90 mg/m². In some embodiments, onvansertib is administered at 15 mg/m². In some embodiments, the topoisomerase I inhibitor is administered at 50-1000 mg/m². In some embodiments, the topoisomerase I inhibitor is administered at 180 mg/m². In some embodiments, the angiogenesis inhibitor is administered at 1-30 mg/kg. In some embodiments, the angiogenesis inhibitor is administered at 5 mg/kg. In some embodiments, a maximum concentration (Cₘₐₓ) of onvansertib in a blood of the subject is from about 100 nmol/L to about 1500 nmol/L after administration. In some embodiments, an area under curve (AUC) of a plot of a concentration of onvanserib in a blood of the subject over time is from about 1000 nmol/L·hour to about 400000 nmol/L·hour after administration. In some embodiments, a time (Tmax) to reach a maximum concentration of onvansertib in a blood of the subject is from about 1 hour to about 5 hours after administration. In some embodiments, an elimination half-life (T_{1/2}) of onvansertib in a blood of the subject is from about 10 hours to about 60 hours after administration.

In some embodiments, the inhibition of progression of the cancer by the topoisomerase I inhibitor and onvansertib is greater than an added inhibition of progression of the cancer caused by the topoisomerase I inhibitor alone plus onvansertib alone. In some embodiments, the inhibition of progression of the cancer by the topoisomerase I inhibitor, the angiogenesis inhibitor and onvansertib is greater than an added inhibition of progression of the cancer caused by (1) the topoisomerase I inhibitor alone, the angiogenesis inhibitor alone and/or a combination of the topoisomerase I inhibitor and the angiogenesis inhibitor; plus (2) onvansertib alone. In some embodiments, the inhibition of progression of the cancer is measured by tumor growth inhibition (TGI). In some embodiments, the TGI resulted by the treatment comprising the topoisomerase I inhibitor and onvansertib is at least 30% higher than the TGI of the topoisomerase I inhibitor alone. In some embodiments, a tumor volume after treatment with the topoisomerase I inhibitor and onvansertib decreases at least 50% relative to the tumor volume with no treatment. In some embodiments, the subject achieves a complete response.

In some embodiments, the subject is a mammal, e.g., human. In some embodiments, the subject has received at least one prior cancer treatment. In some embodiments, the prior cancer treatment does not comprise the use of a topoisomerase I inhibitor, the angiogenesis inhibitor, onvansertib, or a combination thereof. In some embodiments, the subject was in remission for cancer, e.g., complete remission (CR) or in partial remission (PR). In some embodiments, the subject has received a prior topoisomerase I inhibitor treatment. In some embodiments, the subject did not respond to a treatment with the topoisomerase I inhibitor alone. The method can further comprise determining responsiveness of the subject to onvansertib treatment.

Disclosed herein include methods for sensitizing cancer cells to a topoisomerase I inhibitor. For example, the method can comprise: contacting the cancer cells with a composition comprising onvansertib, thereby sensitizing the cancer cells to the topoisomerase I inhibitor.

In some embodiments, contacting the cancer cells with the composition comprising onvansertib occurs in vitro, ex vivo, and/or in vivo. In some embodiments, contacting the cancer cells with the composition comprising onvansertib is in a subject. The method can comprise contacting the cancer cells with the topoisomerase I inhibitor.

The method can comprise evaluating sensitization of the cancer cells to the topoisomerase I inhibitor after being contacted with onvansertib. In some embodiments, evaluating sensitization of the cancer cells to the topoisomerase I inhibitor comprises determining 1) colony forming capacity, 2) number of cancer cells in G2 and/or mitotic stages, 3) phosphorylation of NPM, 4) percentage of cancer cells containing phosphorylated histone H3 (pHH3), 5) percentage of cancer cells expressing cleaved caspase-3 and/or γ-H2AX, 6) expression of γ-H2AX, and/or 7) amount of cleaved caspase-3 and/or cleaved PARP. In some embodiments, the cancer cells have at least one RAS mutation (e.g., HRAS mutation, KRAS mutation and/or NRAS mutation). In some embodiments, the RAS mutation is KRAS mutation and/or NRAS mutation.

The method can further comprise contacting the cancer cells with an angiogenesis inhibitor, e.g., bevacizumab. The method can further comprise contacting the cancer cells with one or more additional therapeutic agents. In some embodiments, the one or more additional therapeutic agents comprise leucovorin, 5-FU or a combination thereof. The topoisomerase I inhibitor can be selected from irinotecan, topotecan, camptothecin, lamellarin D, 9-aminocamptothecin, SN-38, GG-211, DX-8951f, EGCG, genistein, quercetin, resveratrol and a combination thereof. In some embodiments, the topoisomerase I inhibitor is irinotecan.

Disclosed herein include methods for treating cancer. In some embodiments, the method comprises: administrating FOLFIRI, bevacizumab and onvansertib to a subject with cancer, thereby inhibiting progression of the cancer. In some embodiments, FOLFIRI comprises irinotecan, leucovorin, and 5-FU. In some embodiments, the method comprises: administrating onvansertib and a chemotherapy comprising irinotecan to a subject with cancer, thereby inhibiting progression of the cancer, and wherein the subject has been treated with irinotecan and/or the subject is known to be resistant to an irinotecan treatment. In some embodiments, the subject has been treated with irinotecan, the subject is known to be resistant to an irinotecan treatment, or both. In some embodiments, the chemotherapy comprises FOLFIRI and bevacizumab, and wherein FOLFIRI comprises irinotecan, leucovorin, and fluorouracil (5-FU). In some embodiments, the patient is a patient progressing on irinotecan before study entry, a patient who had progressed on irinotecan and were then treated with another therapy prior entering treatment described herein, or a patient on irinotecan treatment prior to study and stable (no progression).

Without being bound by any particular theory, it is believed that onvansertib can sensitize tumors to irinotecan for patients (e.g., for patients who were progressing and had progressed on irinotecan), onvansertib can extend the effect of irinotecan/delay resistance for patients (e.g., for patients who were stable on irinotecan), or both.

Disclosed herein include kits for treating cancer. The kit comprises: onvansertib; and a manual providing instructions for administrating onvansertib with a topoisomerase I inhibitor to a subject for treating cancer. In some embodiments, the subject has been treated with irinotecan and/or the subject is known to be resistant to an irinotecan treatment. The kit can further comprise an angiogenesis inhibitor. In some embodiments, the topoisomerase I inhibitor is irinotecan. In some embodiments, the angiogenesis inhibitor is bevacizumab. The kit can further comprise one or more additional therapeutic agents. In some embodiments, the one or more additional therapeutic agents comprise leucovorin, 5-FU or a combination thereof.

### BRIEF DESCRIPTION OF THE DRAWINGS

FIG. 1 depicts non-limiting exemplary embodiments and data related to workflow of ctDNA analysis.

FIG. 2A-FIG. 2F depict non-limiting exemplary embodiments and data related to tumor volume changes in B8141R (NRAS Q61R) (FIG. 2A), C1177R (KRAS G12C) (FIG. 2B), C1143 (KRAS G12D) (FIG. 2C), B8182 (KRAS G12C) (FIG. 2D), B8086 (KRAS G12V) (FIG. 2E), and C1144 (KRAS G12C) (FIG. 2F), relative to baseline. Means±standard deviation is shown in graphs. An unpaired t-test was used to test the difference in tumor volume change on the last day of treatment between the combination treatment and the most effective control arm. ** indicates p<0.01, and *** indicates p<0.001.

FIG. 3A-FIG. 3F depict non-limiting exemplary embodiments and data related to relative tumor volume of B8141R (NRAS Q61R) (FIG. 3A), C1177R (KRAS G12C) (FIG. 3B), C1143 (KRAS G12D) (FIG. 3C), B8182 (KRAS G12C) (FIG. 3D), B8086 (KRAS G12V) (FIG. 3E), and C1144 (KRAS G12C) (FIG. 3F) on last day of treatment. Means±standard deviation is shown in graphs. An unpaired t-test was used to test the difference in tumor volume change on the last day of treatment between the combination treatment and the most effective control arm. ** indicates p<0.01, and *** indicates p<0.001.

FIG. 4 depicts non-limiting exemplary embodiments and data related to progression-free survival (PFS) of KRAS Responders and Non-Responders.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods for treating cancer. The method can comprise: administrating a topoisomerase I inhibitor (e.g., irinotecan) and onvansertib to a subject with cancer, thereby inhibiting progression of the cancer. The method can comprise: administrating a chemotherapy (e.g., FOLFIRI) and onvansertib to a subject with cancer, thereby inhibiting progression of the cancer. In the methods described herein, the subject can be a subject that has received a topoisomerase I inhibitor therapy and/or is known to be resistant to the topoisomerase I inhibitor therapy. The topoisomerase I inhibitor therapy can be, for example, an irinotecan-based therapy. The method can comprise administrating an angiogenesis inhibitor (e.g., bevacizumab) and/or one or more additional therapeutic agents (e.g., leucovorin, fluorouracil (5-FU) or a combination thereof).

Disclosed herein include methods for sensitizing cancer cells to a topoisomerase I inhibitor (e.g., irinotecan). In some embodiments, the method comprises: contacting the cancer cells with a composition comprising onvansertib, thereby sensitizing the cancer cells to the topoisomerase I inhibitor. The method can comprise evaluating sensitization of the cancer cells to the topoisomerase I inhibitor after being contacted with onvansertib. The method can comprise contacting the cancer cells with the topoisomerase I inhibitor (e.g., irinotecan). The method can further comprise contacting the cancer cells with an angiogenesis inhibitor (e.g., bevacizumab). The method can further comprise contacting the cancer cells with one or more additional therapeutic agents (e.g., leucovorin, 5-FU or a combination thereof).

Disclosed herein include methods for treating cancer. In some embodiments, the method comprises: administrating FOLFIRI, bevacizumab and onvansertib to a subject with cancer, thereby inhibiting progression of the cancer. In some embodiments, FOLFIRI comprises irinotecan, leucovorin, and 5-FU. In some embodiments, the subject has been treated with irinotecan and/or the subject is known to be resistant to an irinotecan treatment.

Disclosed herein include kits comprises: onvansertib; and a manual providing instructions for administrating onvansertib with a topoisomerase I inhibitor (e.g., irinotecan) to a subject for treating cancer. In some embodiments, the subject has been treated with irinotecan and/or the subject is known to be resistant to an irinotecan treatment. The kit can further comprise an angiogenesis inhibitor (e.g., bevacizumab). The kit can further comprise one or more additional therapeutic agents (e.g., leucovorin, 5-FU or a combination thereof).

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, a “subject” refers to an animal that is the object of treatment, observation or experiment. “Animals” include cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that is being treated by a medical professional, such as a Medical Doctor (i.e., Doctor of Allopathic medicine or Doctor of Osteopathic medicine) or a Doctor of Veterinary Medicine, to attempt to cure, or at least ameliorate the effects of, a particular disease or disorder or to prevent the disease or disorder from occurring in the first place. In some embodiments, the patient is a human or an animal. In some embodiments, the patient is a mammal.

As used herein, “administration” or “administering” refers to a method of giving a dosage of a pharmaceutically active ingredient to a vertebrate.

As used herein, a “dosage” refers to the combined amount of the active ingredients (e.g., onvansertib, a topoisomerase I inhibitor (e.g., irinotecan), or a combination of onvansertib and the topoisomerase I inhibitor).

As used herein, a “unit dosage” refers to an amount of therapeutic agent administered to a patient in a single dose.

As used herein, the term “daily dose” or “daily dosage” refers to a total amount of a pharmaceutical composition or a therapeutic agent that is to be taken within 24 hours.

As used herein, the term “delivery” refers to approaches, formulations, technologies, and systems for transporting a pharmaceutical composition or a therapeutic agent into the body of a patient as needed to safely achieve its desired therapeutic effect. In some embodiments, an effective amount of the composition or agent is formulated for delivery into the blood stream of a patient.

As used herein, the term “formulated” or “formulation” refers to the process in which different chemical substances, including one or more pharmaceutically active ingredients, are combined to produce a dosage form. Two or more pharmaceutically active ingredients can be co-formulated into a single dosage form or combined dosage unit, or formulated separately and subsequently combined into a combined dosage unit. A sustained release formulation is a formulation which is designed to slowly release a therapeutic agent in the body over an extended period of time, whereas an immediate release formulation is a formulation which is designed to quickly release a therapeutic agent in the body over a shortened period of time.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to a diseased tissue or a tissue adjacent to the diseased tissue. Carriers or excipients can be used to produce compositions. The carriers or excipients can be chosen to facilitate administration of a drug or pro-drug. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

As used herein, the term “pharmaceutically acceptable salt” refers to any acid or base addition salt whose counter-ions are non-toxic to the patient in pharmaceutical doses of the salts. A host of pharmaceutically acceptable salts are well known in the pharmaceutical field. If pharmaceutically acceptable salts of the compounds of this disclosure are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate, undecanoate, hydrohalides (e.g., hydrochlorides and hydrobromides), sulphates, phosphates, nitrates, sulphamates, malonates, salicylates, methylene-bis-b-hydroxynaphthoates, gentisates, isethionates, di-p-toluoyltartrates, ethanesulphonates, cyclohexylsulphamates, quinates, and the like. Pharmaceutically acceptable base addition salts include, without limitation, those derived from alkali or alkaline earth metal bases or conventional organic bases, such as triethylamine, pyridine, piperidine, morpholine, N-methylmorpholine, ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

As used herein, the term “hydrate” refers to a complex formed by combination of water molecules with molecules or ions of the solute. As used herein, the term “solvate” refers to a complex formed by combination of solvent molecules with molecules or ions of the solute. The solvent can be an organic compound, an inorganic compound, or a mixture of both. Solvate is meant to include hydrate, hemi-hydrate, channel hydrate etc. Some examples of solvents include, but are not limited to, methanol, N,N-dimethylformamide, tetrahydrofuran, dimethylsulfoxide, and water.

As used herein, “therapeutically effective amount” or “pharmaceutically effective amount” refers to an amount of therapeutic agent, which has a therapeutic effect. The dosages of a pharmaceutically active ingredient which are useful in treatment when administered alone or in combination with one or more additional therapeutic agents are therapeutically effective amounts. Thus, as used herein, a therapeutically effective amount refers to an amount of therapeutic agent, which produces the desired therapeutic effect as judged by clinical trial results and/or model animal studies. The therapeutically effective amount will vary depending on the compound, the disease, disorder or condition and its severity and the age, weight, etc., of the mammal to be treated. The dosage can be conveniently administered, e.g., in divided doses up to four times a day or in sustained-release form.

As used herein, the term “treat,” “treatment,” or “treating,” refers to administering a therapeutic agent or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition. As used herein, a “therapeutic effect” relieves, to some extent, one or more of the symptoms of a disease or disorder. For example, a therapeutic effect may be observed by a reduction of the subjective discomfort that is communicated by a subject (e.g., reduced discomfort noted in self-administered patient questionnaire).

As used herein, each of the terms “partial response” and “partial remission” refers to the amelioration of a cancerous state, as measured by, for example, tumor size and/or cancer marker levels, in response to a treatment. In some embodiments, a “partial response” means that a tumor or tumor-indicating blood marker has decreased in size or level by about 50% in response to a treatment. The treatment can be any treatment directed against cancer, including but not limited to, chemotherapy, radiation therapy, hormone therapy, surgery, cell or bone marrow transplantation, and immunotherapy. The size of a tumor can be detected by clinical or by radiological means. Tumor-indicating markers can be detected by means well known to those of skill, e.g., ELISA or other antibody-based tests.

As used herein, each of the terms “complete response” or “complete remission” means that a cancerous state, as measured by, for example, tumor size and/or cancer marker levels, has disappeared following a treatment, including but are not limited to, chemotherapy, radiation therapy, hormone therapy, surgery, cell or bone marrow transplantation, and immunotherapy. The presence of a tumor can be detected by clinical or by radiological means. Tumor-indicating markers can be detected by means well known to those of skill, e.g., ELISA or other antibody-based tests. A “complete response” does not necessarily indicate that the cancer has been cured, however, as a complete response can be followed by a relapse.

As used herein, the term “RAS mutated cancer” refers to a cancer, which is clinically classified as a cancer having one or more mutations in one or more RAS oncogenes. The most common RAS oncogenes are HRAS, NRAS and KRAS. Whether or not a cancer is a RAS mutated cancer can be determined clinically in ways known to the skilled person, such as by examining a tumor biopsy from the patient or in any other suitable way, such as by analyzing RAS mutation status in isolated circulating tumor cells (CTCs). A patient suffering from a cancer that is classified as RAS mutated means that the cancer cells contain one or more RAS mutations that result in constitutively active RAS.

As used herein, the term “angiogenesis inhibitor” refers to an active pharmaceutical ingredient that inhibits angiogenesis. Some angiogenesis inhibitors are VEGF inhibitors. A non-limiting exemplary angiogenesis inhibitor is bevacizumab.

### Cancer

Methods, compositions and kits disclosed herein can be used for treating cancer. Disclosed herein include methods for treating cancer. In some embodiments, the method comprises: administrating a topoisomerase I inhibitor (e.g., irinotecan) and onvansertib to a subject with cancer, thereby inhibiting progression of the cancer. In some embodiments, the subject has received a topoisomerase I inhibitor therapy and/or is known to be resistant to the topoisomerase I inhibitor therapy.

The methods, compositions and kits disclosed herein can be used to various types of cancer, including but are not limited to, colorectal cancer (e.g., metastatic colorectal cancer (mCRC)), melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, lung cancer (e.g., non-small cell lung cancer (NSCLC) and small-cell lung cancer (SCLC)), esophageal cancer, squamous cell carcinoma of the head and neck, liver cancer, ovarian cancer, cervical cancer, thyroid cancer, glioblastoma, glioma, leukemia, lymphoma, and other neoplastic malignancies. Additionally, the disease or condition provided herein includes refractory or recurrent malignancies whose growth can be inhibited using the methods and compositions disclosed herein. In some embodiments, the cancer is carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, or a combination thereof. In some embodiments, the cancer is carcinoma, squamous carcinoma (e.g., cancer of cervical canal, eyelid, tunica conjunctiva, vagina, lung, oral cavity, skin, urinary bladder, tongue, larynx, and gullet), and adenocarcinoma (for example, cancer of prostate, small intestine, endometrium, cervical canal, large intestine, lung, pancreas, gullet, rectum, uterus, stomach, mammary gland, and ovary). In some embodiments, the cancer is bone cancer, breast cancer, brain tumor, central nervous system tumor, colorectal cancer, connective tissue cancer, endometrial cancer, esophageal cancer, gastric cancer, head and neck cancer, kidney cancer, leukemia, liver cancer, lung cancer, lymphoma, myeloma, ovarian cancer, pancreatic cancer, prostate cancer, skin cancer, soft tissue sarcoma, thyroid cancer, or bladder cancer.

The cancer can be a solid tumor, a liquid tumor, or a combination thereof. In some embodiments, the cancer is a solid tumor, including but are not limited to, melanoma, renal cell carcinoma, lung cancer, bladder cancer, breast cancer, cervical cancer, colorectal cancer, gall bladder cancer, laryngeal cancer, liver cancer, thyroid cancer, stomach cancer, salivary gland cancer, prostate cancer, pancreatic cancer, Merkel cell carcinoma, brain and central nervous system cancers, and any combination thereof. In some embodiments, the cancer is a liquid tumor. The cancer can be colorectal cancer, head and neck cancer, lung cancer, intrahepatic cholangiocarcinoma (iCCA), gastric cancer, urothelial cancer, endometrial cancer, cervical cancer, rhabdomyosarcoma, cholangiocarcinoma, glioblastoma, low-grade glioma, ovarian cancer, prostate adenocarcinoma, thyroid carcinoma, endometrial cancer, gallbladder cancer, breast cancer, or a combination thereof.

The cancer can be colorectal cancer. Colorectal cancer and metastatic colorectal cancer (mCRC) are the third most common cancer in men (10% of the total) and the second most common cancer in women (9.2%), with over a hundred and thirty million cases reported worldwide during 2012 (746000 in men and 614000 in women). The geographic incidence of colorectal cancer varies widely throughout the world, and the geographic patterns of males and females are very similar. The incidence in both sexes varies ten-fold worldwide, with the highest estimated incidence in Australia/New Zealand (Australian incidence of 44.8 and 32.2 per 100,000 in men and women, respectively) and the lowest estimated incidence in Western Africa (4.5 and 3.8 per 100,000). The incidence increases with age and is highest in the elderly population (e.g., number of patients per 100000 humans of 60-64 years old: 67.4 humans; 65-69 years old: 95.1 human; 70-74 years old: 127.8 people; and more than or equal to 75 years old: 196.2 human morbidity). About 40%-50% of affected patients develop metastatic disease and more than 50 million deaths due to colorectal cancer are reported annually. In fact, colorectal cancer causes 694,000 deaths (8.5% of the total) only during 2012 Worldwide. The colorectal cancer can be, for example, colorectal adenoma, colon cancer, rectal cancer, colorectal carcinoma, colorectal adenocarcinoma, gastrointestinal carcinoid tumors, gastrointestinal stromal tumors (GISTs), primary colorectal lymphomas, colon leimyosarcomas, rectal leimyosarcomas, colon melanomas, rectal melanomas, colorectal squamous cell carcinoma, and a combination thereof. The cancer can be metastatic colorectal cancer (mCRC). The mCRC can be associated with a RAS mutation.

The cancer can be RAS-mutated cancer, which comprises one or more RAS alterations and/or RAS aberrant activation such as copy number alteration (CNA), single-nucleotide variation (SNV), and gene rearrangement or fusions. The RAS alterations can be in one or more of the RAS isoforms including KRAS, NRAS and/or HRAS. Non-limiting exemplary cancer with RAS alterations include colorectal cancer, breast cancer, lung cancer (non-small cell lung cancer and small cell lung cancer), glioblastomas, head and neck cancers, malignant melanomas, basal cell skin cancer, squamous cell skin cancer, liver cancer, pancreatic cancer, prostate cancer, anal cancer, cervix uteri cancer, bladder cancer, corpus uteri cancer, ovarian cancer, gall bladder cancer, sarcomas, leukemia's (myeloid and lymphatic), lymphomas, myelomatosis, and cholangiocarcinoma.

The cancer can comprise one or more SNVs in one or more of the RAS genes (e.g., KRAS, NRAS and HRAS). In certain embodiments, the RAS mutation involves codons 12, 13, or 61. In certain embodiments, the RAS-mutated cancer is a KRAS-mutated cancer, including, but not limited to, KRASG12C/D/V, KRASG13C/D, or KRASQ61L/H/R. In certain embodiments, the RAS-mutated cancer is an NRAS-mutated cancer, including, but not limited to, NRASQ61R, NRASQ61K, NRASQ61L, or NRASQ61H. In certain embodiments, the RAS-mutated cancer is an HRAS-mutated cancer, including, but not limited to, HRASG12V, HRASQ61R, and HRASG12S.

The RAS mutation can be an HRAS mutation, a KRAS mutation and/or an NRAS mutation. The RAS mutation can be a KRAS mutation and/or an NRAS mutation. The RAS mutation can be in codon 12, codon 13, codon 61 or a combination thereof. The RAS mutation can be selected from KRASG12C, KRASG12D, KRASG12V, KRASG13C, KRASG13D, KRASQ61L, KRASQ61H, KRASQ61R, NRASQ61R, NRASQ61K, NRASQ61L, NRASQ61H, HRASG12V, HRASQ61R, HRASG12S and a combination thereof. The RAS mutation can be NRASQ61R, KRASG12C, KRASG12D, or KRASG12V.

### Topoisomerase I Inhibitors

DNA is normally a coiled double helix of two strands and is periodically uncoiled in the process of replication during cell division or in the process of reading the code to make new proteins. Two enzymes that play the essential role in this uncoiling and recoiling process are topoisomerase I and topoisomerase II. They also play a significant role in fixing DNA damage that occurs as a result of exposure to harmful chemicals or UV rays. The method disclosed herein can comprise administrating an angiogenesis inhibitor to the subject.

There are distinct differences in ways the two enzymes work. Topoisomerase I cuts a single strand of the DNA double helix, while topoisomerase II cuts both strands of DNA, using ATP for fuel. The rest of the process by which the two enzymes work is very similar. The process entails the relaxation of the coil of the two DNA strands, and then after the cuts are made and replication or repair is complete, the strands are paired back together and reform a coil.

The topoisomerase enzymes have been researched as targets for the generation of new cancer treatments because inhibition of the topoisomerase enzymes in a cell results in cell death. Therefore, inhibitors of the topoisomerase enzymes have the ability to kill all cells undergoing DNA replication, transcribing of the DNA for protein production or experiencing repair of DNA damage. Since cancer cells divide much more rapidly than normal cells, the cancer cells will be more sensitive to killing by the topoisomerase inhibitors, though some normal cells with topoisomerase activity will also be killed.

The typical way that both topoisomerase I and topoisomerase II inhibitors work is that the inhibitor binds to the topoisomerase, making the enzyme nonfunctional by preventing the topoisomerase to bind the DNA back together after the DNA has been cut. Therefore, cuts are made to either one strand or both strands of the DNA molecule, which are never repaired, ultimately leading to death of the cell.

Some of the currently known topoisomerase I inhibitors include irinotecan, topotecan, camptothecin, lamellarin D, 9-aminocamptothecin, SN-38, GG-211, DX-8951f, EGCG, genistein, quercetin, and resveratrol. Irinotecan, alone or in combination, is currently clinically used for treating colorectal or metastatic colorectal cancer.

### PLK Inhibitors

Polo-like kinases (PLK) are a family of five highly conserved serine/threonine protein kinases. PLK1 is a master regulator of mitosis and is involved in several steps of the cell cycle, including mitosis entry, centrosome maturation, bipolar spindle formation, chromosome separation, and cytokinesis. PLK1 has been shown to be overexpressed in solid tumors and hematologic malignancies, including AML. PLK1 facilitates homologous recombination (HR) during Double Strand DNA Break (DSB) Repair. PLK1 phosphorylates Rad51 and BRCA1, facilitating their recruitment to DSB sites and thereby HR-mediated DNA repair. PLK1 inhibition induces G2-M-phase arrest with subsequent apoptosis in cancer cells, and has emerged as a promising targeted therapy. Several PLK inhibitors have been studied in clinical trials. In a randomized phase II study of patients with acute myeloid leukemia (AML) who were treatment-naïve yet unsuitable for induction therapy, the pan-PLK inhibitor, volasertib (BI6727), administered intravenously in combination with low-dose cytarabine (LDAC) showed a significant increase in overall survival (OS) when compared with LDAC alone. A subsequent randomized phase III study identified no benefit of the combination of volasertib and LDAC and described an increased risk of severe infections.

Onvansertib (also known as PCM-075 or NMS-1286937) is a selective ATP-competitive PLK1 inhibitor. Biochemical assays demonstrated high specificity of onvansertib for PLK1 among a panel of 296 kinases, including other PLK members. Onvansertib has potent in vitro and in vivo antitumor activity in models of both solid and hematologic malignancies. Onvansertib inhibited cell proliferation at nano-molar concentrations in AML cell lines and tumor growth in xenograft models of AML. In addition, onvansertib significantly increased cytarabine antitumor activity in disseminated models of AML.

A phase I, first-in-human, dose-escalation study of onvansertib in patients with advanced/metastatic solid tumors identified neutropenia and thrombocytopenia as the primary dose-limiting toxicities. These hematologic toxicities were anticipated on the basis of the mechanism of action of the drug and were reversible, with recovery occurring within 3 weeks. The half-life of onvansertib was established between 20 and 30 hours. The oral bioavailability of onvansertib plus its short half-life provide the opportunity for convenient, controlled, and flexible dosing schedules with the potential to minimize toxicities and improve the therapeutic window. Pharmacodynamics and biomarker studies, including baseline genomic profiling, serial monitoring of mutant allele fractions in plasma, and the extent of PLK1 inhibition in circulating blasts, have been performed to identify biomarkers associated with clinical response and are described in PCT Application No. PCT/US2021/013287, the content of which is incorporated herein by reference in its entirety.

As disclosed herein, a combinational therapy using a topoisomerase I inhibitor (e.g., irinotecan) and PLK1 inhibitor (e.g., onvansertib) can result in significantly enhanced efficacy against cancer (e.g., colorectal cancer), causing tumor regression and improved cancer survival. Surprisingly, the resulting tumor regression and cancer survival rate/duration by the combination is more than additive, i.e., superior to the cumulated/added anti-tumor efficacy caused by the topoisomerase I inhibitor (e.g., irinotecan) and the PLK1 inhibitor separately. The PLK1 inhibitor can be onvansertib. Provided herein include methods, compositions and kits for treating cancer in a subject (for example, a human patient suffering from cancer). The method comprises a topoisomerase I inhibitor and onvansertib to a subject with cancer, thereby inhibiting progression of the cancer. In some embodiments, the subject has received a topoisomerase I inhibitor therapy and/or is known to be resistant to the topoisomerase I inhibitor therapy. For example, the topoisomerase I inhibitor and the PLK1 inhibitor can be administrated to a subject with cancer simultaneously, separately, or sequentially. As disclosed herein, the combination treatment using onvansertib and the topoisomerase I inhibitor (e.g., irinotecan) is significantly more effective than the treatment for various cancer using onvansertib and the topoisomerase I inhibitor (e.g., irinotecan) alone, including the treatment for colorectal cancer (e.g., mCRC). It is also expected that the combination treatment using onvansertib and the topoisomerase I inhibitor (e.g., irinotecan) has equivalent or better safety and toxicity profile than the treatment for various cancer using onvansertib and the topoisomerase I inhibitor (e.g., irinotecan) alone.

In some embodiments, the inhibition of cancer progression is not merely additive, but is enhanced or synergistic (that is, the inhibition is greater than the added inhibition of progression caused by the topoisomerase I inhibitor (e.g., irinotecan) alone plus the PLK1 inhibitor alone). The enhanced or synergistic efficacy or inhibition of any combination of the topoisomerase I inhibitor (e.g., irinotecan) and a PLK1 inhibitor (e.g., onvansertib) of the present disclosure can be different in different embodiments. In some embodiments, the enhanced or synergistic efficacy or inhibition of any combination of the topoisomerase I inhibitor and the PLK1 inhibitor of the present disclosure is, is about, is at least, is at least about, is at most, or is at most about, 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or a number or a range between any two of these values, higher than the added inhibition of progression caused by the topoisomerase I inhibitor (e.g., irinotecan) alone plus the PLK1 inhibitor (e.g., onvansertib) alone.

The inhibition of progression of the cancer by the topoisomerase I inhibitor and onvansertib can be greater than an added inhibition of progression of the cancer caused by the topoisomerase I inhibitor alone plus onvansertib alone. The inhibition of progression of the cancer can be measured by tumor growth inhibition (TGI). The TGI resulted by the treatment comprising the topoisomerase I inhibitor and onvansertib can be about, can be at least, can be at least about, can be at most, or can be at most about, 5% (e.g., 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or a number or a range between any two of these values) higher than the TGI of the topoisomerase I inhibitor alone. A tumor volume after treatment with the topoisomerase I inhibitor and onvansertib can decrease about, at least, at least about, at most, or at most about, 5% (e.g., 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or a number or a range between any two of these values) relative to the tumor volume with no treatment.

The progression-free survival (PFS) and percentage of 6-month PFS of the cancer patients receiving the combination treatment of the topoisomerase I inhibitor and onvansertib can be greater than patients receiving the topoisomerase I inhibitor alone and onvansertib alone. The improve of PFS and percentage of 6-month PFS caused by the combination treatment is greater than an added improvement caused by the topoisomerase I inhibitor alone plus onvansertib alone. The PFS can be increased by about, at least, at least about, at most, or at most about, 5% (e.g., 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or a number or a range between any two of these values), relative to the effects caused by the topoisomerase I inhibitor alone, onvansertib alone, or the added improvement caused by the topoisomerase I inhibitor alone plus onvansertib alone. The 6-month PFS can be increased by about, at least, at least about, at most, or at most about, 5% (e.g., 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or a number or a range between any two of these values), relative to the effects caused by the topoisomerase I inhibitor alone, onvansertib alone, or the added improvement caused by the topoisomerase I inhibitor alone plus onvansertib alone.

The method disclosed herein is expected to be effective with various cancer, for example, colorectal cancer, head and neck cancer, lung cancer, intrahepatic cholangiocarcinoma (iCCA), gastric cancer, urothelial cancer, endometrial cancer, cervical cancer, rhabdomyosarcoma, cholangiocarcinoma, glioblastoma, low-grade glioma, ovarian cancer, prostate adenocarcinoma, thyroid carcinoma, endometrial cancer, gallbladder cancer, breast cancer, or a combination thereof.

As described herein, the patient can achieve complete response or partial response after treatment with the topoisomerase I inhibitor and the PLK1 inhibitor. In some embodiments, the patient achieves a complete response. In some embodiments, the patient achieves a partial response. In some embodiments, the subject has received a prior topoisomerase I inhibitor treatment. The prior cancer treatment does not comprise the use of a topoisomerase I inhibitor, onvansertib, or a combination thereof. In some embodiments, the patient did not respond to treatment with only topoisomerase I inhibitor(s). In some embodiments, the subject was in remission for cancer. In some embodiments, the subject in remission for cancer was in complete remission (CR) or in partial remission (PR).

The topoisomerase I inhibitor and the PLK1 inhibitor can be administered to the patient in any manner deemed effective to treat the cancer. The topoisomerase I inhibitor can be administered together with, or separately from, the PLK1 inhibitor. For example, onvansertib and the topoisomerase I inhibitor can be co-administered simultaneously. Onvansertib and the topoisomerase I inhibitor can be administered sequentially. When administered separately, the topoisomerase I inhibitor can be administered before or after the PLK1 inhibitor, or in different administration cycles.

The topoisomerase I inhibitor can be administered through the same route, or a different route from, the PLK1 inhibitor. For example, onvansertib and the topoisomerase I inhibitor can be administered through different routes. Onvansertib and the topoisomerase I inhibitor can be administered through the same route. One or more of onvansertib and the topoisomerase I inhibitor can be administered orally, intraperitoneally, intravenously, subcutaneously, intramuscularly, intrathecally, or by inhalation.

The topoisomerase I inhibitor and the PLK1 inhibitor can each be administered in any schedule, e.g., once or multiple times per day or week; once, twice, three times, four times, five times, six times or seven times (daily) per week; for one or multiple weeks; etc. One or more of onvansertib, and the topoisomerase I inhibitor can be administered in a cycle of at least about 7 days, a cycle of at least about 14 days, a cycle of at least about 21 days, or a cycle of at least about 28 days. Onvansertib, and the topoisomerase I inhibitor can be administered in a cycle of about 28 days. Onvansertib can be administered on at least four days in the cycle. Onvansertib can be administered on days 1-5 and 15-19 in the cycle of 28 days. The topoisomerase I inhibitor can be administered on at least one day in the cycle. The topoisomerase I inhibitor can be administered on days 1 and 15 in the cycle of 28 days. In further embodiments, the patient undergoes at least two cycles of administration.

The topoisomerase I inhibitor (e.g., irinotecan) can be administered to the patient at any appropriate dosage, e.g., a dosage of about, at least or at most 2 mg/kg, 5 mg/kg, 10 mg/kg, 20 mg/kg, or a number between any two of these values. The topoisomerase I inhibitor can be administered at 50-1000 mg/m² (e.g., 50 mg/m², 60 mg/m², 70 mg/m², 80 mg/m², 90 mg/m², 100 mg/m², 200 mg/m², 300 mg/m², 400 mg/m², 500 mg/m², 600 mg/m², 700 mg/m², 800 mg/m², 900 mg/m², 1000 mg/m², or a number or a range between any two of these values). The topoisomerase I inhibitor can be administered at 180 mg/m². The dosage unit based on the body weight (mg/kg) can be converted to another unit (e.g., mg/m²) using a conversion chart such as the body surface area (BSA) conversion chart as will be understood by a person skilled in the art.

Similarly, any PLK1 inhibitor, now known or later discovered, can be used in these methods, including PLK1 inhibitors that are selective for PLK1, and PLK1 inhibitors that also inhibit the activity of other proteins. In some embodiments, the PLK1 inhibitor is a dihydropteridinone, a pyridopyrimidine, a aminopyrimidine, a substituted thiazolidinone, a pteridine derivative, a dihydroimidazo[1,5-f]pteridine, a metasubstituted thiazolidinone, a benzyl styryl sulfone analogue, a stilbene derivative, or a combination thereof. In some of these embodiments, the PLK1 inhibitor is onvansertib, BI2536, Volasertib (BI 6727), GSK461364, AZD1775, CYC140, HMN-176, HMIN-214, rigosertib (ON-01910), MLN0905, TKM-080301, TAK-960 or Ro3280.

In some embodiments, the PLK1 inhibitor is onvansertib. In these embodiments, the onvansertib is administered to the patient at any appropriate dosage, e.g., a dosage of less than 12 mg/m², less than or equal to 24 mg/m², or greater than 24 mg/m². Onvansertib can be administered at 8 mg/m²-90 mg/m² (e.g., 8 mg/m², 9 mg/m², 10 mg/m², 20 mg/m², 30 mg/m², 40 mg/m², 50 mg/m², 60 mg/m², 70 mg/m², 80 mg/m², 90 mg/m², or a number or a range between any two of these values). Onvansertib can be administered at 15 mg/m². Onvansertib can be administered on at least four days in the cycle. In additional embodiments, the onvansertib is administered in a cycle of 28 with onvansertib administration on days 1-5 and 15-19. The combination treatment with onvansertib and the topoisomerase I inhibitor can be administered at the same dose as single treatment with onvansertib or the topoisomerase I inhibitor.

As can be appreciated by one of skill in the art, the amount of co-administration of the topoisomerase I inhibitor and the PLK1 inhibitor, and the timing of co-administration, can depend on the type (species, gender, age, weight, etc.) and condition of the subject being treated and the severity of the disease or condition being treated. The topoisomerase I inhibitor and the PLK1 inhibitor can be formulated into a single pharmaceutical composition, or two separate pharmaceutical compositions. The active ingredients can also be entrapped in microcapsules prepared, for example, by coacervation techniques or by interracial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions.

Methods, compositions, kits and systems disclosed herein can be applied to different types of subjects. For example, the subject can be a subject receiving a cancer treatment, a subject at cancer remission, a subject who has received one or more cancer treatment, or a subject suspected of having cancer. The subject can have a stage I cancer, a stage II cancer, a stage III cancer, and/or a stage IV cancer. The cancer can be colorectal cancer, head and neck cancer, lung cancer, intrahepatic cholangiocarcinoma (iCCA), gastric cancer, urothelial cancer, endometrial cancer, cervical cancer, rhabdomyosarcoma, cholangiocarcinoma, glioblastoma, low-grade glioma, ovarian cancer, prostate adenocarcinoma, thyroid carcinoma, endometrial cancer, gallbladder cancer, breast cancer, or a combination thereof. The cancer can be a RAS-mutated cancer. The methods can further comprise administering an additional therapeutic intervention to the subject. The additional therapeutic intervention can comprise a different therapeutic intervention than administering the PLK1 inhibitor and the topoisomerase I inhibitor, including but not limited to an angiogenesis inhibitor, an antibody, an adoptive T cell therapy, a chimeric antigen receptor (CAR) T cell therapy, an antibody-drug conjugate, a cytokine therapy, a cancer vaccine, a checkpoint inhibitor, a radiation therapy, surgery, a chemotherapeutic agent, or any combination thereof. The therapeutic intervention can be administered at any time of the treatment, for example at a time when the subject has an early-stage cancer. The therapeutic intervention can be more effective that if the therapeutic intervention were to be administered to the subject at a later time. Without being bound to any particular theory, it is believed that the PLK1 inhibitor (e.g., onvansertib) can sensitize cells (e.g., cancer cells) to topoisomerase I inhibitor treatment to achieve effective cancer treatment.

### Additional Cancer Therapeutics or Therapy

Methods, compositions and kits disclosed herein can be used for treating cancer, for example colorectal cancer. In some embodiments, a method for treating cancer comprises administrating a topoisomerase I inhibitor and a PLK1 inhibitor (e.g., onvansertib) to a subject (e.g., a patient) in need thereof. The method can comprise administering a therapeutically effective amount of the topoisomerase I inhibitor and a therapeutically effective amount of the PLK1 inhibitor. The treatment can also comprise administration of at least one additional cancer therapeutics or cancer therapy. The treatment can comprise administration a therapeutically effective amount of at least one additional cancer therapeutics or cancer therapy. The topoisomerase I inhibitor and the cancer therapeutics or cancer therapy can, for example, co-administered simultaneously or sequentially. The PLK1 inhibitor (e.g., onvansertib) and the cancer therapeutics or cancer therapy can, for example, co-administered simultaneously or sequentially.

A number of antibodies (mAb) are approved or are in development for use in the treatment of cancers. Nonlimiting examples of antibodies suitable in treating cancer, such as NSCLC, are selected from: Atezolizumab (marketed as Tecentriq®, target PD-1/PDL-1), Avelumab (marketed as Bavencio®, target PD-1/PDL-1), Balstilimab (target PD-1/PDL-1), Bevacizumab (marketed as Avastin®, target VEGF), Camrelizumab (target PD-1/PDL-1), Cemiplimab (marketed as Libtayo®, target PD-1/PDL-1), Cetuximab (marketed as Erbitux®, target EGFR), Dostarlimab (target PD-1/PDL-1), Durvalumab (marketed as Imfinzi®, target PD-1/PDL-1), Necitumumab (marketed as Portrazza®, target EGFR), Nimotuzumab (marketed as Theraloc®, target EGFR), Nivolumab (marketed as Opdivo®, target PD-1/PDL-1), Panitumumab (marketed as Vectibix®, target EGFR), Pembrolizumab (marketed as Keytruda®, target PD-1/PDL-1), Prolgolimab (marketed as Forteca®, target PD-1/PDL-1), Racotumomab (marketed as Vaxira®, target NeuGcGM3), Ramucirumab (marketed as Cymraza®, target VEGF), Ranibizumab (marketed as Lucentis®, target VEGF), Retifanlimab (target PD-1/PDL-1), Sintilimab (marketed as Tyvyt®, target PD-1/PDL-1), Tislelizumab (target PD-1/PDL-1), and Toripalimab (marketed as Tuoyi, target PD-1/PDL-1).

Non-limiting examples of angiogenesis inhibitor include aflibercept, axitinib, bevacizumab, cabozantinib, everolimus (RAD001), lenvatinib, pazopanib, ponatinib, ramucirumab, ranibizumab, regorafenib, sorafenib, sunitinib, temsirolimus (CCI-779), vandetanib, or any combination thereof. In one embodiment, the angiogenesis inhibitor is an antibody selected from bevacizumab, ramucirumab, and ranibizumab, and combinations thereof. Bevacizumab is a monoclonal antibody that functions as an angiogenesis inhibitor. It works by slowing the growth of new blood vessels by inhibiting vascular endothelial growth factor A (VEGF-A), in other words anti-VEGF therapy.

The angiogenesis inhibitor can be administered to the patient in any manner deemed effective to treat the cancer. The angiogenesis inhibitor can be administered together with, or separately from, the topoisomerase I inhibitor and/or the PLK1 inhibitor. For example, the angiogenesis inhibitor can be co-administered simultaneously with onvansertib and/or the topoisomerase I inhibitor. The angiogenesis inhibitor can be administered sequentially with onvansertib and/or the topoisomerase I inhibitor. When administered separately, the angiogenesis inhibitor can be administered before or after one of the topoisomerase I inhibitor and the PLK1 inhibitor, or in different administration cycles.

The angiogenesis inhibitor can be administered through the same route, or different route from, one of the topoisomerase I inhibitor and the PLK1 inhibitor. The angiogenesis inhibitor can be administered orally, intraperitoneally, intravenously, subcutaneously, intramuscularly, intrathecally, or by inhalation.

The angiogenesis inhibitor can be administered in any schedule, e.g., once or multiple times per day or week; once, twice, three times, four times, five times, six times or seven times (daily) per week; for one or multiple weeks; etc. The angiogenesis inhibitor can be administered in a cycle of at least about 7 days, a cycle of at least about 14 days, a cycle of at least about 21 days, or a cycle of at least about 28 days. The angiogenesis inhibitor can be administered in a cycle of about 28 days. The angiogenesis inhibitor can be administered on at least one day in the cycle. The angiogenesis inhibitor can be administered on days 1 and 15 in the cycle of 28 days. In further embodiments, the patient undergoes at least two cycles of administration.

FOLFIRI is a known chemotherapy regimen approved for human use comprising the combined administration of folinic acid, 5-fluorouracil (5-FU) and irinotecan. FOLFIRI can be, for example, administered in up to 12 two-week cycles. FOLFIRI combines drugs, each with a different mechanism of action and favorably with synergistic effects, causing the death of cancer cells. 5-Fluorouracil (5-FU, CAS registry number 51-21-8) is an anti-metabolite, which principally inhibits thymidylate synthase and, thus, blocks the synthesis of thymidine. 5-Fluorouracil has been used in the treatment of colon cancer, esophageal cancer, stomach cancer, pancreatic cancer, breast cancer, and cervical cancer. Folinic acid, also known as leucovorin (CAS registry number 58-05-9), stabilizes the complex between 5-fluorouracil and thymidylate synthase, increasing the cytotoxicity of 5-fluorouracil. In one embodiment, folinic acid is L-folinic acid (N-[4-[[[(6S)-2-amino-5-formyl-3,4,5,6,7,8-hexahydro-4-oxo-6-pteridinyl]methyl]amino]benzoyl]-L-glutamic acid). In some embodiments, folinic acid is the calcium salt of L-folinic acid. Folinic acid can also comprise a mixture two or more stereoisomers. Irinotecan (CAS Number 97682-44-5) is a cytotoxic component, which is a semi-synthetic derivative of the alkaloid camptothecin and inhibits topoisomerase I, resulting in inhibition of DNA replication and transcription and which has been used in the treatment of colon cancer and small cell lung cancer.

The method disclosed herein can further comprise administrating to the subject one or more additional therapeutic agents. The one or more additional therapeutic agents can comprise leucovorin, fluorouracil or a combination thereof. Disclosed herein include methods for treating cancer. In some embodiments, the method comprises: administrating FOLFIRI, bevacizumab and onvansertib to a subject with cancer, thereby inhibiting progression of the cancer. FOLFIRI can comprise irinotecan, leucovorin, and 5-FU. In some embodiments, the subject has been treated with irinotecan and/or the subject is known to be resistant to an irinotecan treatment.

One or more additional therapeutic agents can be administered to the patient in any manner deemed effective to treat the cancer. The one or more additional therapeutic agents can be administered together with, or separately from, the topoisomerase I inhibitor and/or the PLK1 inhibitor. For example, the additional therapeutic agent(s) can be co-administered simultaneously with onvansertib and/or the topoisomerase I inhibitor. The additional therapeutic agent(s) can be administered sequentially with onvansertib and/or the topoisomerase I inhibitor. When administered separately, the additional therapeutic agent(s) can be administered before or after one of the topoisomerase I inhibitor (e.g., irinotecan) and the PLK1 inhibitor (e.g., onvansertib), or in different administration cycles.

The one or more additional therapeutic agents can be administered through the same route, or different route from, one of the topoisomerase I inhibitor and the PLK1 inhibitor. The additional therapeutic agent(s) can be administered orally, intraperitoneally, intravenously, subcutaneously, intramuscularly, intrathecally, or by inhalation.

The one or more additional therapeutic agents can be administered in any schedule, e.g., once or multiple times per day or week; once, twice, three times, four times, five times, six times or seven times (daily) per week; for one or multiple weeks; etc. The additional therapeutic agent(s) can be administered in a cycle of at least about 7 days, a cycle of at least about 14 days, a cycle of at least about 21 days, or a cycle of at least about 28 days. The additional therapeutic agent(s) can be administered in a cycle of about 28 days. The additional therapeutic agent(s) can be administered on at least one day in the cycle. The additional therapeutic agent(s) can be administered on days 1 and 15 in the cycle of 28 days. The patient can, for example, undergoes at least two cycles of administration (e.g., two, three, four, five, or more cycles of administration).

In some embodiments, the subject has received and failed one or more prior irinotecan-based therapies. In some embodiments, the subject has received a prior topoisomerase I inhibitor treatment. The prior cancer treatment does not comprise the use of a topoisomerase I inhibitor, the angiogenesis inhibitor, onvansertib, or a combination thereof. In some embodiments, the patient did not respond to treatment with only topoisomerase I inhibitor(s).

The inhibition of progression of the cancer by the topoisomerase I inhibitor, the angiogenesis inhibitor and onvansertib can be greater than an added inhibition of progression of the cancer caused by 1) the topoisomerase I inhibitor alone, the angiogenesis inhibitor alone and/or a combination of the topoisomerase I inhibitor and the angiogenesis inhibitor; plus 2) onvansertib alone.

### Dosing and Pharmacokinetics

The treatment of the present disclosure can comprise administration of a PLK1 inhibitor (e.g., onvansertib) for a desired duration in one or more cycles of treatment, and administration of one or more topoisomerase I inhibitors, one or more angiogenesis inhibitor and one or more additional therapeutic agents.

Daily administration of a topoisomerase I inhibitor (e.g., irinotecan) can be at, or be at about, 50-1000 mg/m² (e.g., 50 mg/m², 60 mg/m², 70 mg/m², 80 mg/m², 90 mg/m², 100 mg/m², 200 mg/m², 300 mg/m², 400 mg/m², 500 mg/m², 600 mg/m², 700 mg/m², 800 mg/m², 900 mg/m², 1000 mg/m², or a number or a range between any two of these values). The daily dose of the topoisomerase I inhibitor (e.g., irinotecan) can be adjusted (e.g., increased or decreased with the range) during the treatment of the subject. The daily administration of the topoisomerase I inhibitor can be at different amounts on different days or during different weeks. For example, the treatment can comprise daily administration of the topoisomerase I inhibitor (e.g., irinotecan) at 180 mg/m² for two days (e.g., Days 1 and 15) during a 28-day cycle.

The treatment of the present disclosure can comprise administration of the topoisomerase I inhibitor (e.g., irinotecan) for a desired duration in a cycle. The administration of the topoisomerase I inhibitor can be daily or with break(s) between days of administrations. The break can be, for example, 1 day, 2 days, 3 days, 4 days, 5 days, 6 days, 7 days, 8 days, 9 days, 10 days, 11 days, 12 days, 13 days, 14 days, or more. The administration can be once, twice, three times, four times, or more on a day when the topoisomerase I inhibitor (e.g., irinotecan) is administered to the patient. The administration can be, for example, once every two days, every three days, every four days, every five days, every six days, or every seven days. The length of the desired duration can vary, for example, 1, 2, 3, 4, 5, 6, 7, 8, 9, 10, 11, 12, 13, 14, 15, 16, 17, 18, 19, 20, 21, 22, 23, 24, 25, 26, 27, 28, or more days. Each cycle of treatment can have various lengths, for example, at least 14 days, 15 days, 16 days, 17 days, 18 days, 19 days, 20 days, 21 days, 22 days, 23 days, 24 days, 25 days, 26 days, 27 days, 28 days, or more. For example, a single cycle of the treatment can comprise administration of the topoisomerase I inhibitor (e.g., irinotecan) for one day, two days, three days, four days, five days, six days, seven days, eight days, nine days, ten days, eleven days, twelve days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, or more in a cycle (e.g., in a cycle of at least 21 days (e.g., 21 to 28 days)). The treatment can comprise administration of the topoisomerase I inhibitor (e.g., irinotecan) for, or for at least, one day, two days, three days, four days, five days, six days, seven days, eight days, nine days, ten days, eleven days, twelve days, thirteen days, fourteen days, fifteen days, sixteen days, seventeen days, eighteen days, nineteen days, twenty days, or a range between any two of these values, in a cycle (e.g., a cycle of at least 21 days (e.g., 21 to 28 days)). The administration of the topoisomerase I inhibitor (e.g., irinotecan) in a single cycle of the treatment can be continuous or with one or more intervals (e.g., one day or two days of break). In some embodiments, the treatment comprises administration of the topoisomerase I inhibitor (e.g., irinotecan) for two days in a cycle of 21 to 28 days.

In some embodiments, the topoisomerase I inhibitor (e.g., irinotecan) is administered to the subject in need thereof on two days (e.g., Days 1 and 15) during a 28-day cycle. The two days can be, for example, a continuous daily administration for two days (e.g., Days 1-2 or Days 15-16), or two separated days (e.g., Days 1 and 15). Depending on the needs of inhibition/reversion of cancer progression in the subject, the subject can receive one, two, three, four, five, six, or more cycles of treatment.

A maximum concentration (Cₘₐₓ) of the topoisomerase I inhibitor (e.g., irinotecan) in a blood of the subject (during the treatment or after the treatment) after administration when the topoisomerase I inhibitor is administered alone or in combination with the PLK1 inhibitor can be from about 300 nmol/L to about 15000 nmol/L. For example, the Cₘₐₓ of the topoisomerase I inhibitor (e.g., irinotecan) in a blood of the subject when the topoisomerase I inhibitor is administered alone or in combination with the PLK1 inhibitor can be, or be about, 300 nmol/L, 400 nmol/L, 500 nmol/L, 600 nmol/L, 700 nmol/L, 800 nmol/L, 900 nmol/L, 1000 nmol/L, 2000 nmol/L, 3000 nmol/L, 4000 nmol/L, 5000 nmol/L, 6000 nmol/L, 7000 nmol/L, 8000 nmol/L, 9000 nmol/L, 10000 nmol/L, 11000 nmol/L, 12000 nmol/L, 13000 nmol/L, 14000 nmol/L, 15000 nmol/L, a range between any two of these values, or any value between 300 nmol/L to about 15000 nmol/L.

An area under curve (AUC) of a plot of a concentration of the topoisomerase I inhibitor (e.g., irinotecan) in a blood of the subject over time (e.g., AUC₀₋₂₄ for the first 24 hours after administration) after administration when the topoisomerase I inhibitor is administered alone or in combination with the PLK1 inhibitor can be from about 1,000 nmol/L·hour to about 4,000,000 nmol/L·hour. For example, the AUC of a plot of a concentration of the topoisomerase I inhibitor (e.g., irinotecan) in a blood of the subject over time (e.g., AUC₀₋₂₄ for the first 24 hours after administration) when the topoisomerase I inhibitor (e.g., irinotecan) is administered alone or in combination with the PLK1 inhibitor can be, or be about, 1,000 nmol/L·hour, 2,000 nmol/L·hour, 3,000 nmol/L·hour, 4,000 nmol/L·hour, 5,000 nmol/L·hour, 6,000 nmol/L·hour, 7,000 nmol/L·hour, 8,000 nmol/L·hour, 9,000 nmol/L·hour, 10,000 nmol/L·hour, 20,000 nmol/L·hour, 30,000 nmol/L·hour, 40,000 nmol/L·hour, 50,000 nmol/L·hour, 60,000 nmol/L·hour, 70,000 nmol/L·hour, 80,000 nmol/L·hour, 90,000 nmol/L·hour, 100,000 nmol/L·hour, 200,000 nmol/L·hour, 300,000 nmol/L·hour, 400,000 nmol/L·hour, 500,000 nmol/L·hour, 600,000 nmol/L·hour, 700,000 nmol/L·hour, 800,000 nmol/L·hour, 900,000 nmol/L·hour, 1,000,000 nmol/L·hour, 2,000,000 nmol/L·hour, 3,000,000 nmol/L·hour, 4,000,000 nmol/L·hour, a range between any two of these values, or any value between 1,000 nmol/L·hour to about 4,000,000 nmol/L·hour.

A time (Tₘₐₓ) to reach a maximum concentration of the topoisomerase I inhibitor (e.g., irinotecan) in a blood of the subject when the topoisomerase I inhibitor (e.g., irinotecan) is administered alone or in combination with the PLK1 inhibitor can be from about 1 hours to 10 hours. For example, the time (Tₘₐₓ) to reach a maximum concentration of the topoisomerase I inhibitor (e.g., irinotecan) in a blood of the subject when the topoisomerase I inhibitor is administered alone or in combination with the PLK1 inhibitor can be, or be about, 1 hour, 1.5 hours, 2 hours, 2.5 hours, 3 hours, 4 hours, 5 hours, 6 hours, 7 hours, 8 hours, 9 hours, 10 hours, a range between any two of these values, or any value between 3 hours and 10 hours.

An elimination half-life (T_{1/2}) of the topoisomerase I inhibitor (e.g., irinotecan) in a blood of the subject when the topoisomerase I inhibitor (e.g., irinotecan) is administered alone or in combination with the PLK1 inhibitor can be from about 15 hours to about 60 hours. For example, the elimination half-life (T_{1/2}) of the topoisomerase I inhibitor (e.g., irinotecan) in a blood of the subject when the topoisomerase I inhibitor is administered alone or in combination with the PLK1 inhibitor can be, or be about, 15 hours, 20 hours, 25 hours, 30 hours, 35 hours, 40 hours, 45 hours, 50 hours, 55 hours, 60 hours, a range between any two of these values, or any value between 15 hours and 60 hours.

The treatment of the present disclosure can comprise administration of a PLK1 inhibitor (e.g., onvansertib) for a desired duration in a cycle. The administration of the PLKs inhibitor can be daily or with break(s) between days of administrations. The break can be, for example, 1 day, 2 days, 3 days, 4 days, 5 days, 6 days, 7 days, 8 days, 9 days, 10 days, 11 days, 12 days, 13 days, 14 days, or more. The administration can be once, twice, three times, four times, or more on a day when the PLK1 inhibitor is administered to the patient. The administration can be, for example, once every two days, every three days, every four days, every five days, every six days, or every seven days. The length of the desired duration can vary, for example, 1, 2, 3, 4, 5, 6, 7, 8, 9, 10, 11, 12, 13, 14, 15, 16, 17, 18, 19, 20, 21, 22, 23, 24, 25, 26, 27, 28, or more days. Each cycle of treatment can have various lengths, for example, at least 14 days, 15 days, 16 days, 17 days, 18 days, 19 days, 20 days, 21 days, 22 days, 23 days, 24 days, 25 days, 26 days, 27 days, 28 days, or more. For example, a single cycle of the treatment can comprise administration of the PLK1 inhibitor (e.g., onvansertib) for four days, five days, six days, seven days, eight days, nine days, ten days, eleven days, twelve days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, or more in a cycle (e.g., in a cycle of at least 21 days (e.g., 21 to 28 days)). The treatment can comprise administration of the PLK1 inhibitor (e.g., onvansertib) for, or for at least, four days, five days, six days, seven days, eight days, nine days, ten days, eleven days, twelve days, thirteen days, fourteen days, fifteen days, sixteen days, seventeen days, eighteen days, nineteen days, twenty days, or a range between any two of these values, in a cycle (e.g., a cycle of at least 21 days (e.g., 21 to 28 days)). The administration of the PLK1 inhibitor (e.g., onvansertib) in a single cycle of the treatment can be continuous or with one or more intervals (e.g., one day or two days of break). In some embodiments, the treatment comprises administration of the PLK1 inhibitor (e.g., onvansertib) for five days in a cycle of 21 to 28 days.

In some embodiments, the PLK1 inhibitor (e.g., onvansertib) is administered to the subject in need thereof on twenty days (e.g., Days 1-10 and 15-24) during a 28-day cycle. The twenty days can be, for example, a continuous daily administration for ten days (e.g., Days 1-10) and another continuous daily administration (e.g., Days 15-24) for ten days, or a continuous daily administration for four sets of five days (e.g., Days 1-5, 8-12, 15-19, and 22-26). In some embodiments, for example when the patient is identified to have low tolerance to the PLK1 inhibitor (e.g., onvansertib), the PLK1 inhibitor is administered to the subject in need thereof on ten days (e.g., Days 1-5 and 15-19) during a 28-day cycle. The ten days can be, for example, a continuous daily administration for ten days (e.g., Days 1-10) or two continuous daily admiration for five days each (e.g., Days 1-5 and Days 15-19). In some embodiments, the PLK1 inhibitor (e.g., onvansertib) is administered to the subject in need thereof daily throughout the whole cycle (e.g., daily for 28 days in a cycle of 28 days). Depending on the needs of inhibition/reversion of cancer progression in the subject, the subject can receive one, two, three, four, five, six, or more cycles of treatment.

The treatment can comprise administration of the PLK1 inhibitor (e.g., onvansertib) at, or at about, 8 mg/m²-90 mg/m², for example, as a daily dose. For example, the treatment can comprise daily administration of the PLK1 inhibitor (e.g., onvansertib) at, or at about, 8 mg/m²-90 mg/m² (e.g., 8 mg/m², 9 mg/m², 10 mg/m², 20 mg/m², 30 mg/m², 40 mg/m², 50 mg/m², 60 mg/m², 70 mg/m², 80 mg/m², 90 mg/m², or a number or a range between any two of these values). The daily dose of the PLK1 inhibitor (e.g., onvansertib) can be adjusted (e.g., increased or decreased with the range) during the treatment, or during a single cycle (e.g., the first cycle, the second cycle, the third cycle, and a subsequent cycle) of the treatment, for the subject. In some embodiments, the PLK inhibitor (e.g., onvansertib) is administered at 12 mg/m² on twenty days (e.g., Days 1-10 and 15-24) during a 28-day cycle. In some embodiments, the PLK inhibitor (e.g., onvansertib) is administered at 15 mg/m² on ten days (e.g., Days 1-5 and 15-19) during a 28-day cycle. In some embodiments, the PLK inhibitor (e.g., onvansertib) is administered at 8 mg/m² or 10 mg/m² everyday (e.g., Days 11-28) during a 28-day cycle. The daily dose of the PLK1 inhibitor (e.g., onvansertib) can be adjusted (e.g., increased or decreased with the range) during the treatment, or during a single cycle (e.g., the first cycle, the second cycle, the third cycle, and a subsequent cycle) of the treatment, for the subject.

A maximum concentration (Cₘₐₓ) of the PLK1 inhibitor (e.g., onvansertib) in a blood of the subject (during the treatment or after the treatment) when the PLK1 inhibitor is administered alone or in combination with the topoisomerase I inhibitor (e.g., irinotecan) can be from about 100 nmol/L to about 1500 nmol/L. For example, the Cₘₐₓ of the PLK1 inhibitor (e.g., onvansertib) in a blood of the subject when the PLK1 inhibitor is administered alone or in combination with the topoisomerase I inhibitor can be, or be about, 100 nmol/L, 200 nmol/L, 300 nmol/L, 400 nmol/L, 500 nmol/L, 600 nmol/L, 700 nmol/L, 800 nmol/L, 900 nmol/L, 1000 nmol/L, 1100 nmol/L, 1200 nmol/L, 1300 nmol/L, 1400 nmol/L, 1500 nmol/L, a range between any two of these values, or any value between 200 nmol/L to 1500 nmol/L.

An area under curve (AUC) of a plot of a concentration of the PLK1 inhibitor (e.g., onvansertib) in a blood of the subject over time (e.g., AUC₀₋₂₄ for the first 24 hours after administration) when the PLK1 inhibitor is administered alone or in combination with the topoisomerase I inhibitor can be from about 1000 nmol/L·hour to about 400000 nmol/L·hour. For example, the AUC of a plot of a concentration of the PLK1 inhibitor (e.g., onvansertib) in a blood of the subject over time (e.g., AUC₀₋₂₄ for the first 24 hours after administration) when the PLK1 inhibitor is administered alone or in combination with the topoisomerase I inhibitor can be, or be about, 1000 nmol/L·hour, 5000 nmol/L·hour, 10000 nmol/L·hour, 15000 nmol/L·hour, 20000 nmol/L·hour, 25000 nmol/L·hour, 30000 nmol/L·hour, 35000 nmol/L·hour, 40000 nmol/L·hour, a range between any two of these values, or any value between 1000 nmol/L·hour and 400000 nmol/L·hour.

A time (Tₘₐₓ) to reach a maximum concentration of the PLK1 inhibitor (e.g., onvansertib) in a blood of the subject when the PLK1 inhibitor is administered alone or in combination with the topoisomerase I inhibitor can be from about 1 hour to about 5 hours. For example, the time (Tₘₐₓ) to reach a maximum concentration of the PLK1 inhibitor (e.g., onvansertib) in a blood of the subject when the PLK1 inhibitor is administered alone or in combination with the topoisomerase I inhibitor can be, or be about, 1 hour, 1.5 hours, 2 hours, 2.5 hours, 3 hours, 3.5 hours, 4 hours, 4.5 hours, 5 hours, a range between any two of these values, or any value between 1 hour and 5 hours.

An elimination half-life (T_{1/2}) of the PLK1 inhibitor (e.g., onvansertib) in a blood of the subject when the PLK1 inhibitor is administered alone or in combination with the topoisomerase I inhibitor can be from about 10 hours to about 60 hours. For example, the elimination half-life (T_{1/2}) of the PLK1 inhibitor (e.g., onvansertib) in a blood of the subject when the PLK1 inhibitor is administered alone or in combination with the topoisomerase I inhibitor can be, or be about, 10 hours, 15 hours, 20 hours, 25 hours, 30 hours, 35 hours, 40 hours, 45 hours, 50 hours, 55 hours, 60 hours, a range between any two of these values, or any value between 10 hours and 60 hours.

Daily administration of an angiogenesis inhibitor (e.g., bevacizumab) can be at, or be at about, 1-30 mg/kg (e.g., 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, or a number or a range between any two of these values). The daily dose of the angiogenesis inhibitor (e.g., bevacizumab) can be adjusted (e.g., increased or decreased with the range) during the treatment of the subject. The daily administration of the angiogenesis inhibitor can be at different amounts on different days or during different weeks. For example, the treatment can comprise daily administration of the angiogenesis inhibitor (e.g., bevacizumab) at 5 mg/kg for two days (e.g., Days 1 and 15) during a 28-day cycle.

The method of the present disclosure can comprise administration of the angiogenesis inhibitor for a desired duration in a cycle. The administration of the angiogenesis inhibitor can be daily or with break(s) between days of administrations. The break can be, for example, 1 day, 2 days, 3 days, 4 days, 5 days, 6 days, 7 days, 8 days, 9 days, 10 days, 11 days, 12 days, 13 days, 14 days, or more. The administration can be once, twice, three times, four times, or more on a day when the angiogenesis inhibitor is administered to the patient. The administration can be, for example, once every two days, every three days, every four days, every five days, every six days, or every seven days. The length of the desired duration can vary, for example, 1, 2, 3, 4, 5, 6, 7, 8, 9, 10, 11, 12, 13, 14, 15, 16, 17, 18, 19, 20, 21, 22, 23, 24, 25, 26, 27, 28, or more days. Each cycle of treatment can have various lengths, for example, at least 14 days, 15 days, 16 days, 17 days, 18 days, 19 days, 20 days, 21 days, 22 days, 23 days, 24 days, 25 days, 26 days, 27 days, 28 days, or more. For example, a single cycle of the treatment can comprise administration of the angiogenesis inhibitor for one day, two days, three days, four days, five days, six days, seven days, eight days, nine days, ten days, eleven days, twelve days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, or more in a cycle (e.g., in a cycle of at least 21 days (e.g., 21 to 28 days)). The treatment can comprise administration of the angiogenesis inhibitor for, or for at least, one day, two days, three days, four days, five days, six days, seven days, eight days, nine days, ten days, eleven days, twelve days, thirteen days, fourteen days, fifteen days, sixteen days, seventeen days, eighteen days, nineteen days, twenty days, or a range between any two of these values, in a cycle (e.g., a cycle of at least 21 days (e.g., 21 to 28 days)). The administration of the angiogenesis inhibitor in a single cycle of the treatment can be continuous or with one or more intervals (e.g., one day or two days of break). In some embodiments, the treatment comprises administration of the angiogenesis inhibitor for two days in a cycle of 21 to 28 days.

In some embodiments, the angiogenesis inhibitor is administered to the subject in need thereof on two days (e.g., Days 1 and 15) during a 28-day cycle. The twenty days can be, for example, a continuous daily administration for two days (e.g., Days 1-2 or Days 15-16) for two days, or two separated days (e.g., Days 1 and 15). Depending on the needs of inhibition/reversion of cancer progression in the subject, the subject can receive one, two, three, four, five, six, or more cycles of treatment.

Daily administration of the additional therapeutic agent(s) (e.g., leucovorin and/or 5-fluorouracil) can be at, or be at about, 50-2000 mg/m² (e.g., 50 mg/m², 60 mg/m², 70 mg/m², 80 mg/m², 90 mg/m², 100 mg/m², 200 mg/m², 300 mg/m², 400 mg/m², 500 mg/m², 600 mg/m², 700 mg/m², 800 mg/m², 900 mg/m², 1000 mg/m², 2000 mg/m², or a number or a range between any two of these values). The daily dose of the additional therapeutic agent(s) can be adjusted (e.g., increased or decreased with the range) during the treatment of the subject. The daily administration of the additional therapeutic agent(s) can be at different amounts on different days or during different weeks. For example, the treatment can comprise daily administration of the additional therapeutic agent(s) at 400 mg/m² for two days (e.g., Days 1 and 15) during a 28-day cycle.

The method of the present disclosure can comprise administration of the additional therapeutic agent(s) (e.g., leucovorin and/or 5-fluorouracil) for a desired duration in a cycle. The administration of the additional therapeutic agent(s) can be daily or with break(s) between days of administrations. The break can be, for example, 1 day, 2 days, 3 days, 4 days, 5 days, 6 days, 7 days, 8 days, 9 days, 10 days, 11 days, 12 days, 13 days, 14 days, or more. The administration can be once, twice, three times, four times, or more on a day when the additional therapeutic agent(s) (e.g., leucovorin and/or 5-fluorouracil) is administered to the patient. The administration can be, for example, once every two days, every three days, every four days, every five days, every six days, or every seven days. The length of the desired duration can vary, for example, 1, 2, 3, 4, 5, 6, 7, 8, 9, 10, 11, 12, 13, 14, 15, 16, 17, 18, 19, 20, 21, 22, 23, 24, 25, 26, 27, 28, or more days. Each cycle of treatment can have various lengths, for example, at least 14 days, 15 days, 16 days, 17 days, 18 days, 19 days, 20 days, 21 days, 22 days, 23 days, 24 days, 25 days, 26 days, 27 days, 28 days, or more. For example, a single cycle of the treatment can comprise administration of the additional therapeutic agent(s) (e.g., leucovorin and/or 5-fluorouracil) for one day, two days, three days, four days, five days, six days, seven days, eight days, nine days, ten days, eleven days, twelve days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, or more in a cycle (e.g., in a cycle of at least 21 days (e.g., 21 to 28 days)). The treatment can comprise administration of the additional therapeutic agent(s) (e.g., leucovorin and/or 5-fluorouracil) for, or for at least, one day, two days, three days, four days, five days, six days, seven days, eight days, nine days, ten days, eleven days, twelve days, thirteen days, fourteen days, fifteen days, sixteen days, seventeen days, eighteen days, nineteen days, twenty days, or a range between any two of these values, in a cycle (e.g., a cycle of at least 21 days (e.g., 21 to 28 days)). The administration of the additional therapeutic agent(s) (e.g., leucovorin and/or 5-fluorouracil) in a single cycle of the treatment can be continuous or with one or more intervals (e.g., one day or two days of break). In some embodiments, the treatment comprises administration of the additional therapeutic agent(s) (e.g., leucovorin and/or 5-fluorouracil) for two days in a cycle of 21 to 28 days.

In some embodiments, the additional therapeutic agent(s) (e.g., leucovorin and/or 5-fluorouracil) is administered to the subject in need thereof on two days (e.g., Days 1 and 15) during a 28-day cycle. The twenty days can be, for example, a continuous daily administration for two days (e.g., Days 1-2 or Days 15-16) for two days, or two separated days (e.g., Days 1 and 15). Depending on the needs of inhibition/reversion of cancer progression in the subject, the subject can receive one, two, three, four, five, six, or more cycles of treatment.

### Methods for Sensitizing Cancer Cells

The methods, compositions and kits disclosed herein can also be used to sensitize cancer cells to a topoisomerase I inhibitor (e.g., irinotecan). In some embodiments, the method comprises: contacting the cancer cells with a composition comprising onvansertib, thereby sensitizing the cancer cells to the topoisomerase I inhibitor. Contacting cancer cells with the composition can occur in vitro, ex vivo, in vivo, or in any combination. In some embodiments, cancer cells are contacted with the composition in a cell culture. In some embodiments, contacting cancer cells with the composition is in a subject's body. The subject can be a mammal, for example a human. The subject can be, for example, a subject that did not respond to, or is known to be resistant to, topoisomerase I inhibitors alone. The subject can be, for example, a subject that had prior treatment with one of the topoisomerase I inhibitors. The cancer cells can have at least one RAS mutation. The RAS mutation can be HRAS mutation, KRAS mutation and/or NRAS mutation. The RAS mutation can be KRAS mutation and/or NRAS mutation. In some embodiments, the method comprises determining the response of the subject and/or the cancer cells to the topoisomerase I inhibitors.

The sensitization of the cancer cells can increase the responsiveness of the cancer cells to the topoisomerase I inhibitors by, or by about, 1%, 5%, 10%, 15%, 20%, 25%, 30%, 35%, 40%, 45%, 50%, 55%, 60%, 65%, 70%, 75%, 80%, 85%, 90%, 95%, or a range between any two of these values. The sensitization of the cancer cells can increase the responsiveness of the cancer cells to the topoisomerase I inhibitors by at least, or by at least about, 1%, 5%, 10%, 15%, 20%, 25%, 30%, 35%, 40%, 45%, 50%, 55%, 60%, 65%, 70%, 75%, 80%, 85%, 90%, 95%, or a range between any two of these values. The increase of the responsiveness of the cancer cells is, in some embodiments, relative to the untreated cancer cells. The sensitization of the cancer cells can increase the responsiveness of the subject having the cancer cells to the topoisomerase I inhibitors by, or by about, 1%, 5%, 10%, 15%, 20%, 25%, 30%, 35%, 40%, 45%, 50%, 55%, 60%, 65%, 70%, 75%, 80%, 85%, 90%, 95%, or a range between any two of these values. The sensitization of the cancer cells can increase the responsiveness of the subject having the cancer cells to the topoisomerase I inhibitors by at least, or by at least about, 1%, 5%, 10%, 15%, 20%, 25%, 30%, 35%, 40%, 45%, 50%, 55%, 60%, 65%, 70%, 75%, 80%, 85%, 90%, 95%, or a range between any two of these values. The increase of the responsiveness of the subject having the cancer cells is, in some embodiments, relative to the subjects untreated with the composition.

The method can also comprise contacting the cancer cells with the topoisomerase I inhibitor, e.g., irinotecan, topotecan, camptothecin, lamellarin D, 9-aminocamptothecin, SN-38, GG-211, DX-8951f, EGCG, genistein, quercetin, resveratrol and a combination thereof. The topoisomerase I inhibitor can be irinotecan. The method can comprise contacting the cancer cells with an angiogenesis inhibitor (e.g., bevacizumab). The method can further comprise contacting the cancer cells with one or more additional therapeutic agents. The additional therapeutic agent(s) can be or comprise leucovorin, fluorouracil (5-FU) or a combination thereof.

The method can comprise evaluating sensitization of the cancer cells to the topoisomerase I inhibitor after being contacted with onvansertib. Evaluating sensitization of the cancer cells to the topoisomerase I inhibitor can comprise determining 1) colony forming capacity, 2) number of cancer cells in G2 and/or mitotic stages, 3) phosphorylation of NPM, 4) percentage of cancer cells containing phosphorylated histone H3 (pHH3), 5) percentage of cancer cells expressing cleaved caspase-3 and/or γ-H2AX, 6) expression of γ-H2AX, and/or 7) amount of cleaved caspase-3 and/or cleaved PARP.

The sensitization of the cancer cells can, for example, reduce the colony forming capacity of the cancer cells by, by at least, or by at least about, 20%, 25%, 30%, 35%, 40%, 45%, 50%, 55%, 60%, 65%, 70%, 75%, 80%, 85%, 90%, 95%, or a range between any two of these values. The decrease of the colony-forming capacity of the cancer cells is, in some embodiments, relative to the cancer cells untreated with the composition. The sensitization of the cancer cells can increase the relative number of cells in G2 and/or mitotic stages by at least 1.5 folds, 2 folds, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values. The sensitization of the cancer cells can increase the expression of mitotic markers, such as phosphorylated nucleophosmin (NPM) on Threonine 199, in the cancer cells by at least 1.5 folds, 2 folds, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values. The sensitization of the cancer cells can increase the percentage of cancer cells expressing phosphorylated histone H3 (pHH3) by at least 1.5 folds, 2 folds, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values. The sensitization of the cancer cells can increase the percentage of cancer cells expressing cleaved caspase-3 by at least 1.5 folds, 2 folds, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values. The sensitization of the cancer cells can increase the expression of the apoptotic markers, such as cleaved caspase-3 and cleaved PARP, by at least 1.5 folds, 2 folds, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values. The sensitization of the cancer cells can increase the expression of DNA damage markers, such as γ-H2AX, by at least 1.5 folds, 2 folds, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values. The sensitization of the cancer cells can increase the percentage of cancer cells expressing γ-H2AX by at least 1.5 folds, 2 folds, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values. The increase of the relative number of cells in G2 and/or mitotic stages, of the expression of phosphorylated NPM (Thr199), cleaved caspase 3, cleaved PARP and yH2AX, of the percentage of cancer cells expressing pHH3, cleaved caspase-3 and γ-H2AX is, in some embodiments, relative to those in the cancer cells or subjects untreated with the composition.

The method can comprise determining sensitization of the cancer cells to the topoisomerase I inhibitors after being contacted with the PLK1 inhibitor. The method can comprise contacting the cancer cells with the topoisomerase I inhibitors concurrently and/or after being contacted with the PLK1 inhibitor.

### Methods for Determining Responsiveness to the Treatment

The method, compositions, kits and systems disclosed herein can further comprise determining responsiveness of the subject to PLK1 inhibitor (e.g., onvansertib) treatment. The methods, compositions, kits and systems can be used to guide the combination treatment, provide combination treatment recommendations, reduce or avoid unnecessary ineffective combination treatment for patients. Circulating tumor DNA (ctDNA) can be analyzed to predict/determine clinical outcome for cancer treatment using a combination of an topoisomerase I inhibitor and a PLK1 inhibitor of the present disclosure, monitor the combination treatment, predict/determine responsiveness of a subject to the combination treatment, determine cancer status in a subject, improve the combination treatment outcome, guide combination treatment, provide combination treatment recommendations, and/or to reduce or avoid ineffective combination treatment. ctDNA can be analyzed to predict/determine clinical outcome for cancer treatment, monitor cancer treatment, predict/determine responsiveness of a subject to a cancer treatment, determine cancer status in a subject, improve cancer treatment outcome, guide cancer treatment, provide treatment recommendations, and/or to reduce or avoid ineffective cancer treatment. Such analysis of ctDNA has been described in PCT/US2021/013287, the content of which is incorporated herein by reference in its entirety.

A method of determining responsiveness of a subject to a combination treatment comprising a topoisomerase I inhibitor and a PLK1 inhibitor of the disclosure can comprise, for example, analyzing ctDNA of a subject with cancer, the subject is undergoing a treatment and/or has received the combination treatment, thereby determining the responsiveness of the subject to the combination treatment. In some embodiments, determining the responsiveness of the subject comprises determining if the subject is a responder of the treatment, if the subject is or is going to be in CR, or if the subject is or is going to be in partial remission (PR). For example, analyzing ctDNA can comprise detecting variant allele frequency in the ctDNA in a first sample obtained from the subject at a first time point, detecting variant allele frequency in the ctDNA obtained from the subject at one or more additional time points in one or more additional samples, and determining the difference of the variant allele frequency in ctDNA between the first and at least one of the one or more additional samples, a decrease in the variant allele frequency in at least one of the additional samples relative to the first sample indicates the subject as responsive to the cancer treatment.

The first time point can be prior or immediately prior to the combination treatment, and at least one of the one or more additional time points are at the end of or after at least a cycle of the combination treatment. The cycle of the combination treatment can be the first cycle of the combination treatment. The first time point can be prior or immediately prior to a first cycle of the combination treatment, and the one or more additional time points can be at the end of or after a second cycle of the combination treatment.

The first cycle of the combination treatment can be immediately prior to the second cycle of the combination treatment. The method can comprise continuing the combination treatment to the subject if the subject is indicated as responsive to the combination treatment. The method can comprise discontinuing the combination treatment to the subject and/or starting a different combination treatment to the subject if the subject is not indicated as responsive to the combination treatment.

The variant allele frequency in ctDNA can be determined, for example, by total mutation count in the ctDNA in each of the first sample and one or more additional samples, or by the mean variant allele frequency in each of the first sample and one or more additional samples. The variant allele frequency can be mutant allelic frequency (MAF) for a driver mutation of the cancer (e.g., ovarian cancer, breast cancer, prostate cancer, colorectal cancer, pancreatic cancer, or a combination thereof). The variant allele frequency can be MAF for one or more driver mutations of the cancer (e.g., ovarian cancer, breast cancer, prostate cancer, colorectal cancer, pancreatic cancer, or a combination thereof). Log₂(C₁/C₀)<a MAF threshold indicates a decrease in ctDNA MAF, in which C₀ is ctDNA MAF in the first sample and C₁ is ctDNA MAF in one of the additional samples. The MAF threshold can be, or can be about, 0.01 to −0.10. The MAF threshold can be, or can be about, 0.06. The MAF threshold can be, or can be about, 0.05.

The first sample can comprise ctDNA from the subject before treatment, and the one of additional samples can comprise ctDNA from the subject after treatment. The driver mutation can be a mutation in one of the below 75 genes ABL1, ANKRD26, ASXL1, ATRX, BCOR, BCORL1, BRAF, BTK, CALR, CBL, CBLB, CBLC, CCND2, CDC25C, CDKN2A, CEBPA, CSF3R, CUX1, CXCR4, DCK, DDX41, DHX15, DNMT3A, ETNK1, ETV6, EZH2, FBXW7, FLT3, GATA1, GATA2, GNAS, HRAS, IDH1, IDH2, IKZF1, JAK2, JAK3, KDM6A, KIT, KMT2A, KRAS, LUC7L2, MAP2K1, MPL, MYC, MYD88, NF1, NOTCHI, NPM1, NRAS, PDGFRA, PH1F6, PPM1D, PTEN, PTPN11, RAD21, RBBP6, RPS14, RUNX1, SETBP1, SF3B1, SH2B3, SLC29A1, SMC1A, SMC3, SRSF2, STAG2, STAT3, TET2, TP53, U2AF1, U2AF2, WT1, XPO1, and ZRSR2. At least one of the one or more the driver mutations can be a mutation in the 75 genes. One or more the driver mutations can be mutations in the 75 genes.

The driver mutation or at least one of the one or more driver mutations can be in a gene selected from TP53, ASXL1, DNMT3A, NRAS, SRSF2, TET2, SF3B1, FLT3, FLT3 ITD, IDH2, NPM1, RUNX1, CDKN2A, KRAS, STAG2, CALR, CBL, CSF3R, DDX41, GATA2, JAK2, PHF6, and SETBP1. The driver mutation or at least one of the one or more driver mutations can be in a gene selected from DNMT3A, TET2, NPM1, SRSF2, NRAS, CDKN2A, SF3B1, FLT3, ASXL1, SRSF2, IDH2, NRAS, and SF3B1. The driver mutation or at least one of the one or more driver mutations can be in RAS (e.g., HRAS, KRAS and/or NRAS). The method can comprises determining variant allele frequency in ctDNA, PBMCs and/or BMMCs of the subject.

The ctDNA can be analyzed using, for example, polymerase chain reaction (PCR), next generation sequencing (NGS), and/or droplet digital PCR (ddPCR). The sample disclosed herein can be derived from, for example, whole blood of the subject, plasma of the subject, serum of the subject, or a combination thereof. The ctDNA can be from whole blood of the subject, plasma of the subject, serum of the subject, or a combination thereof.

Responders refer to subjects with at least, or at least about, 30% (e.g., 30%, 40%, 50%, 60%, 70%, 80%, 90%, 100%, or a number or a range between any of these values) decrease in MAF for one or more driver mutations (e.g., RAS mutation) after one cycle of treatment. Non-responders refer to subjects with less than, or less than about, 30% (e.g., 30%, 40%, 50%, 60%, 70%, 80%, 90%, 100%, or a number or a range between any of these values) decrease in MAF for one or more driver mutations (e.g., RAS mutation) after one cycle of treatment. The PFS and percentage of 6-month PFS of the responders can be greater than the PFS and percentage of 6-month PFS of the non-responders. The PFS of the responders can be about, at least, at least about, at most, or at most about, 5% (e.g., 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or a number or a range between any two of these values), greater than the non-responders. The 6-month PFS of the responders can be about, at least, at least about, at most, or at most about, 5% (e.g., 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or a number or a range between any two of these values), greater than the non-responders.

### Compositions and Kits

Disclosed herein include compositions and kits for treating cancer. The kit comprises: onvansertib; and a manual providing instructions for administrating onvansertib with a topoisomerase I inhibitor to a subject for treating cancer. In some embodiments, the subject has been treated with the topoisomerase I inhibitor and/or the subject is known to be resistant to a topoisomerase I inhibitor treatment. The kit can further comprise an angiogenesis inhibitor. The topoisomerase I inhibitor can be irinotecan. The angiogenesis inhibitor can be bevacizumab. The kit can further comprise one or more additional therapeutic agents. The additional therapeutic agent(s) can be or comprise leucovorin, fluorouracil (5-FU) or a combination thereof.

The cancer can be, for example, colorectal cancer, head and neck cancer, lung cancer, intrahepatic cholangiocarcinoma (iCCA), gastric cancer, urothelial cancer, endometrial cancer, cervical cancer, rhabdomyosarcoma, cholangiocarcinoma, glioblastoma, low-grade glioma, ovarian cancer, prostate adenocarcinoma, thyroid carcinoma, endometrial cancer, gallbladder cancer, breast cancer, or a combination thereof. The colorectal cancer can be selected from colorectal adenoma, colon cancer, rectal cancer, colorectal carcinoma, colorectal adenocarcinoma, gastrointestinal carcinoid tumors, gastrointestinal stromal tumors (GISTs), primary colorectal lymphomas, colon leiomyosarcomas, rectal leiomyosarcomas, colon melanomas, rectal melanomas, colorectal squamous cell carcinoma, and a combination thereof. The cancer is metastatic colorectal cancer (mCRC). The mCRC can be associated with a RAS mutation.

In some embodiments, the instructions comprise instructions for co-administrating the PLK inhibitor and the topoisomerase I inhibitor simultaneously. In some embodiments, the instructions comprise instructions for co-administrating the PLK inhibitor and the topoisomerase I inhibitor sequentially. In some embodiments, the instructions comprise instructions for administering of the PLK1 inhibitor orally. In some embodiments, the instructions comprise instructions for administrating the topoisomerase I inhibitor intraperitoneally.

In some embodiments, the instructions comprise instructions the subject has received a prior topoisomerase I inhibitor treatment. In some embodiments, the instructions comprise instructions the subject did not respond to treatment with the topoisomerase I inhibitor alone. In some embodiments, the instructions comprise instructions the subject is known to be resistant to a topoisomerase I inhibitor therapy.

In some embodiments, the instructions comprise instructions the subject has received at least one prior treatment for the cancer. In some embodiments, the prior treatment does not comprise the use of a topoisomerase I inhibitor, a PLK inhibitor, or both. In some embodiments, the instructions comprise instructions the subject was in remission for the cancer, e.g., complete remission (CR), or in partial remission (PR).

In some embodiments, the instructions comprise instructions for administering each of the topoisomerase I inhibitor and the PLK1 inhibitor to the subject in a cycle. In some embodiments, the instructions comprise instructions for administering the topoisomerase I inhibitor, the PLK1 inhibitor, or both in a cycle of at least 7 days. In some embodiments, each cycle of treatment is at least about 21 days. In some embodiments, each cycle of treatment is from about 21 days to about 28 days. In some embodiments, each cycle of treatment is at least about 28 days. In some embodiments, the instructions comprise instructions for administering the PLK1 inhibitor on at least four days in the cycle. In some embodiments, the instructions comprise instructions for not administering the PLK1 inhibitor on at least one day in the cycle. In some embodiments, the instructions comprise instructions for administrating the topoisomerase I inhibitor at least one day in the cycle. In some embodiments, the instructions comprise instructions for administrating the topoisomerase I inhibitor and the PLK1 inhibitor for at least two cycles.

In some embodiments, the topoisomerase I inhibitor is selected irinotecan, topotecan, camptothecin, lamellarin D, 9-aminocamptothecin, SN-38, GG-211, DX-8951f, EGCG, genistein, quercetin, resveratrol, and a combination thereof.

In some embodiments, the PLK1 inhibitor is selective and/or specific for PLK1. In some embodiments, the PLK1 inhibitor is a dihydropteridinone, a pyridopyrimidine, a aminopyrimidine, a substituted thiazolidinone, a pteridine derivative, a dihydroimidazo[1,5-f]pteridine, a metasubstituted thiazolidinone, a benzyl styryl sulfone analogue, a stilbene derivative, or any combination thereof. In some embodiments, the PLK1 inhibitor is onvansertib, BI2536, Volasertib (BI 6727), GSK461364, AZD1775, CYC140, HMN-176, HMN-214, rigosertib (ON-01910), MLN0905, TKM-080301, TAK-960 or Ro3280. In some embodiments, the PLK1 inhibitor is onvansertib. In some embodiments, the topoisomerase I inhibitor is irinotecan, and the PLK1 inhibitor is onvansertib.

In some embodiments, the instructions comprise instructions for administering the PLK1 inhibitor at 8 mg/m²-90 mg/m². In some embodiments, the instructions comprise instructions for administering the topoisomerase I inhibitor at 50-1000 mg/m². In some embodiments, the instructions comprise instructions for administering the angiogenesis inhibitor at 1-30 mg/kg.

### EXAMPLES

Some aspects of the embodiments discussed above are disclosed in further detail in the following examples, which are not in any way intended to limit the scope of the present disclosure.

### Materials and Methods

The following experimental materials and methods were used for Examples 1 and 2 described below.

### Combination Treatment of Onvansertib and Irinotecan in Mice (Preclinical)

Six RAS-mutant patient derived xenograft (PDX) models, including 5 KRAS-mutant and 1 NRAS-mutant (Table 1), were used to test the combination of onvansertib and irinotecan. PDX models had either intrinsic resistance to irinotecan (n=4) or acquired resistance (n=2). Acquired resistance was developed by continuous treatment of PDX models with irinotecan until the point of clear progression. Irinotecan-resistant tumors were then passaged to an additional generation.

TABLE 1
DESCRIPTION OF PDX MODELS

| PDX | | | | | | |
|---|---|---|---|---|---|---|
| model | B8141R | C1177R | C1143 | B8086 | B8182 | C1144 |
| RAS | NRAS | KRAS | KRAS | KRAS | KRAS | KRAS |
| mutation | Q61R | G12C | G12D | G12V | G12C | G12C |
| Iri- | Acquired | Acquired | Intrinsic | Intrinsic | Intrinsic | Intrinsic |
| resistance | | | | | | |

Nude mice were inoculated with low passage PDXs. Once tumors reached 150-350 mm³, mice were randomized and treated for up to 21 days with vehicle, onvansertib (oral, 60 mg/kg, daily), irinotecan (intraperitoneal, 40 mg/kg, weekly) or the combination of onvansertib and irinotecan. Tumor volumes and mice body weights were measured twice a week. Tumor volume changes were calculated with the formula ΔV=100%×((Vt−V₀)/V₀). Tumor growth inhibition (TGI) was calculated with the formula TGI (%)=(1−rTi/rVi)×100, in which rTi is the mean relative tumor volume of the treatment group and rVi is the mean relative tumor volume of vehicle group on the same day.

### Combination Treatment of Onvansertib and Irinotecan/Bevacizumab in Human Patients (Clinical)

An Expanded Access Program (EAP) was opened to provide access to the combination therapy of onvansertib and FOLFIRI/bevacizumab to KRAS-mutated mCRC patients, who had failed or progressed on standard-of-care, including irinotecan (NCT04446793). Patients were treated with onvansertib in combination with FOLFIRI/bevacizumab as described in Table 2. In Table 2, optional administration (e.g., Leucovorin and/or 5-FU (bolus)) was given based on institutional guidelines and Investigator discretion.

TABLE 2
DOSES OF ONVANSERTIB, FOLFIRI
AND BEVACIZUMAB IN THE EAP

| Drug | Dose | | Frequency |
|---|---|---|---|
| Bevacizumab | 5 | mg/kg | Days 1 and 15 of each 28-day cycle |
| Irinotecan | 180 | mg/m² | Days 1 and 15 of each 28-day cycle |
| Leucovorin | 400 mg/m² | | Days 1 and 15 of each 28-day cycle |
| | (optional) | | |
| 5-FU (bolus) | 400 mg/m² | | Days 1 and 15 of each 28-day cycle |
| | (optional) | | |
| 5-FU (infusion) | 400 | mg/m² | Days 1 and 15 of each 28-day cycle |
| Onvansertib | 15 | mg/m² | Days 1-5 and 15-19 of each 28-day |
| | | | cycle |

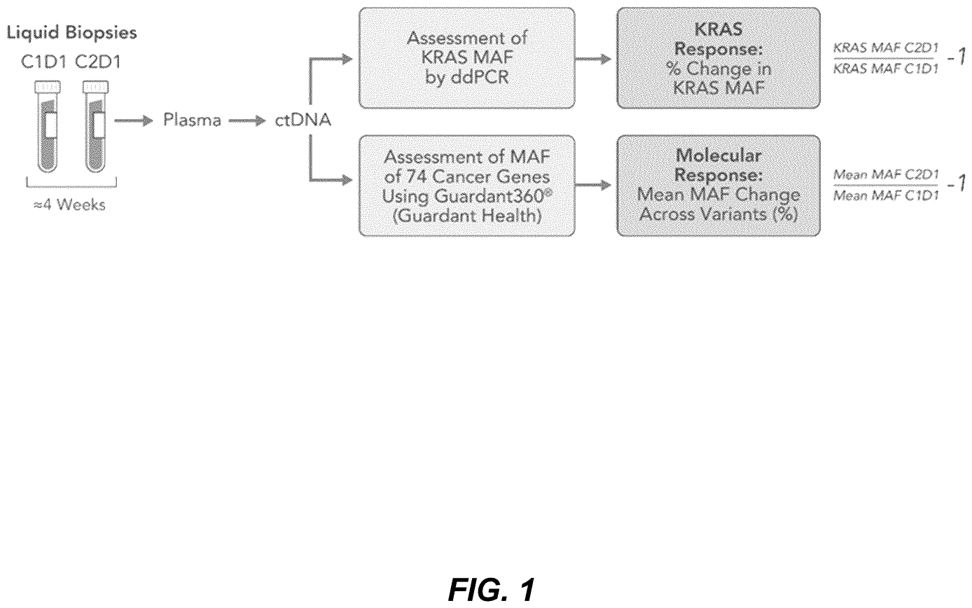

Blood samples were collected from patients into Cell-Free DNA BCT® blood tubes (Streck) at baseline (Pre-dose Cycle 1 Day 1, C1D1) and after 1 cycle of treatment (Pre-dose Cycle 2 Day 1, C2D1) and processed 24-48 hours after collection at Cardiff Oncology's laboratories as shown in FIG. 1. Plasma was separated from whole blood by centrifugation. Circulating tumor DNA (ctDNA) was isolated from plasma. Plasma KRAS mutant allelic frequency (MAF) was assessed by digital droplet PCR (ddPCR). Change in KRAS MAF was calculated as [(KRAS MAF C2D1/KRAS MAF C1D1)-1]%. C1D1 and C2D1 ctDNA samples were also analyzed with the Guardant360® assay (Guardant Health Inc.), a next-generation sequencing (NGS)-based liquid biopsy covering 74 cancer genes, and measuring mean MAF of somatic single-nucleotide variant (SNVs), insertions/deletions, and gene fusions. Mean MAF change across variants was calculated as: [(Mean MAF C2D1/Mean MAF C1D1)-1]%.

### Example 1

### The Anti-Tumor Effects of the Combination Treatment of Onvansertib and Irinotecan in Mice

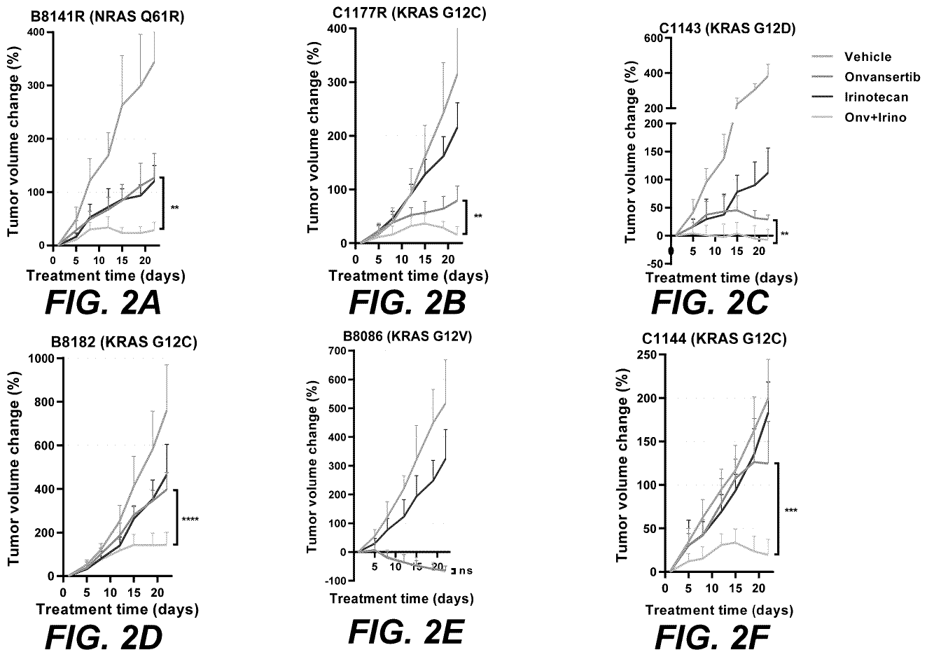

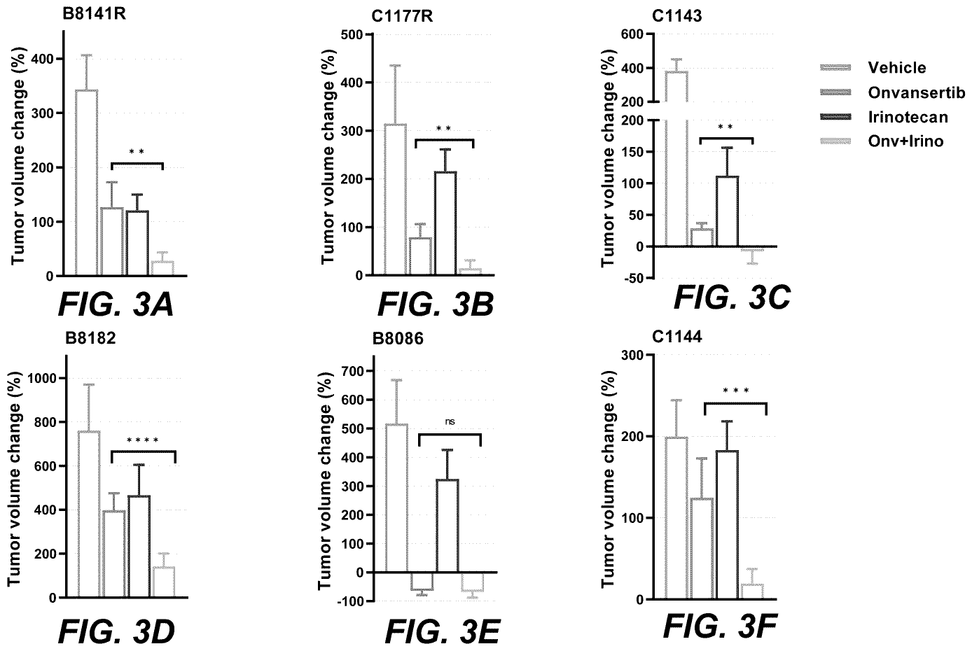

As expected, PDX models were resistant to irinotecan, with a tumor size increase of at least 100% at the end of the treatment compared to baseline (FIG. 2A-FIG. 2F and FIG. 3A-FIG. 3F). The combination of onvansertib and irinotecan showed anti-tumor activity in the RAS-mutated PDX models with acquired and intrinsic resistance to irinotecan, with TGI>80% in all models (Table 3). Importantly, the combination showed significant increased anti-tumor activity compared to onvansertib single agent in 5 of the 6 models (FIG. 2A-FIG. 2F and FIG. 3A-FIG. 3F), indicating that onvansertib re-sensitized tumors to irinotecan treatment. Altogether, these data demonstrate that the combination of onvansertib and irinotecan was an effective combination therapy in RAS-mutated PDX models and that onvansertib sensitized tumors to irinotecan.

TABLE 3
TUMOR GROWTH INHIBITION AT END OF TREATMENT

| Treatment | B8141 | C1177 | C1143 | B8182 | B8086 | C1144 |
|---|---|---|---|---|---|---|
| Onvansertib | 63.0 | 74.9 | 92.6 | 47.7 | 112.4 | 22.5 |
| (Onv) | | | | | | |
| Irinotecan | 64.9 | 31.5 | 70.7 | 38.7 | 37.3 | 16.9 |
| (Irin) | | | | | | |
| Onv + Irin | 91.7 | 95.2 | 102.0 | 81.2 | 113.2 | 85.4 |

This example shows that the combination of onvansertib and irinotecan was an effective combination in RAS-mutated PDX models with acquired or intrinsic resistance to irinotecan. The combination had significantly increased anti-tumor activity compared to the single agent of onvansertib, indicating that the use of onvansertib helped to overcome irinotecan-resistance.

### Example 2

### The Anti-Tumor Effects of the Combination Treatment of Onvansertib and Irinotecan/Bevacizumab in Human Patients

Between July 2020 and June 2021, 51 patients were enrolled in the EAP. Among them, 43 (84%) patients had received irinotecan in a prior line of treatment. As of Aug. 5, 2022, EAP patients with prior irinotecan treatment had a progression-free survival (PFS) of 4.04 months [Confidence interval (CI): 2.96-8.38] and a 6-month PFS of 37.3% [CI: 24.9-55.8].

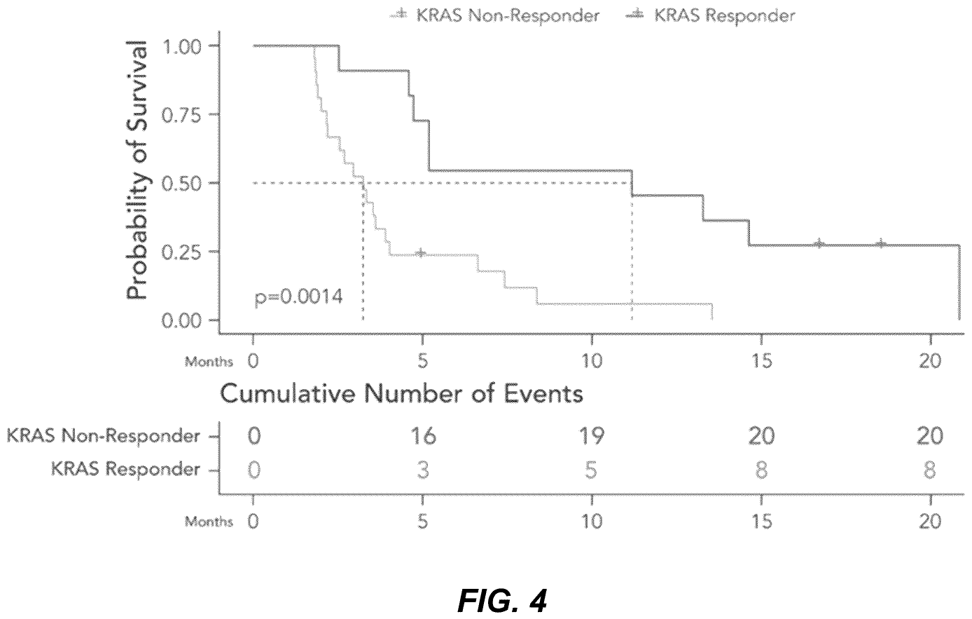

In the Phase 1b/2 clinical study of onvansertib in combination with FOLFIRI/bevacizumab, patients with a ≥90% decrease in MAF of a single target (e.g., KRAS detected with ddPCR) or multi-genes (74-gene panel, detected with NGS) after 1 cycle of treatment, had significantly higher overall response rate and longer PFS than patients with less than 90% decrease in MAF. These data supported the use of early MAF changes in liquid biopsies as a response biomarker to the combination therapy of onvansertib and FOLFIRI/bevacizumab in clinical studies. Therefore, the association between early changes in ctDNA and clinical benefit in the EAP was assessed. Thirty-two EAP patients with prior irinotecan treatment, were evaluated for KRAS response (i.e., had C1D1 and C2D1 plasma samples and detectable baseline KRAS MAF). Eleven (34%) patients were determined to be KRAS Responders (i.e., ≥90% decrease in KRAS MAF after 1 treatment cycle). The KRAS Responders had significantly longer PFS and higher 6-month PFS than KRAS Non-responders (p=0.0014) (Table 4 and FIG. 4).

TABLE 4
EFFICACY OF THE COMBINATION OF ONVANSERTIB
AND FOLFIRI/BEVACIZUMAB IN KRAS RESPONDERS
AND NON-RESPONDERS

| | | | All Patients | |
|---|---|---|---|---|
| | | | Evaluable | All Patients |
| | KRAS | KRAS Non- | for KRAS | with Prior |
| | Responder | Responder | Response | Irinotecan |
| Patients (n) | 11 (34%) | 21 (66%) | 32 | 43 |
| Median PFS | 11.18 | 3.25 | 3.98 | 4.04 |
| [CI] (months) | [5.19-NR] | [2.20-6.64] | [3.25-7.43] | [2.96-8.38] |
| 6-Month PFS | 54.5 | 23.8 | 33.9 | 37.3 |
| [CI] (%) | [31.8-93.6] | [11.1-51.2] | [20.8-55.2] | [24.9-55.8] |

Similarly, changes in mean MAF were tested using the NGS-based Guardant360® assay and could predict clinical benefit of EAP patients who had received prior irinotecan treatment. Molecular Responders were defined as patients with a ≥90% decrease in mean MAF after 1 treatment cycle. Twenty-one EAP patients with prior-irinotecan were evaluated for Molecular Response and 7 (33%) patients were identified as Molecular Responders. Molecular Responders had significantly longer PFS and higher 6-month PFS than Molecular Non-responders (p=0.013) (Table 5).

TABLE 5
EFFICACY OF THE COMBINATION OF ONVANSERTIB AND FOLFIRI/BEVACIZUMAB
IN MOLECULAR RESPONDERS AND NON-RESPONDERS

| | | | | | All Patients | | | |
|---|---|---|---|---|---|---|---|---|
| | | | | | Evaluated for | | All Patients | |
| | Molecular | | Molecular | | Molecular | | with Prior | |
| | Responder | | Non-Responder | | Response | | Irinotecan | |
| Patients (n) | 7 | (33%) | 14 | (67%) | 21 | | 43 | |
| Median PFS [CI] (months) | 13.28 | [4.73-NR] | 3.77 | [3.35-7.43] | 4.60 | [3.55-13.3] | 4.04 | [2.96-8.38] |
| 6-Month PFS [CI] (%) | 57.1 | [30.1-100.0] | 21.4 | [7.9-58.4] | 33.3 | [18.2-61.0] | 37.3 | [24.9-55.8] |

KRAS-mutated mCRC patients with prior irinotecan treatment showed a clinical benefit to onvansertib and FOLFIRI/bevacizumab combination treatment, particularly in patients with early ctDNA decreases. All together this data supported the combination use of onvansertib and FOLFIRI/bevacizumab in RAS-mutated mCRC patients who progressed on standard-of-care, including patients refractory or developing resistant to irinotecan, and the utility of assessing early changes in ctDNA to identify patients who are more likely to benefit from therapy.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of treating cancer, the method comprising: administrating a topoisomerase I inhibitor and onvansertib to a subject with cancer, thereby inhibiting progression of the cancer, wherein the subject has received a topoisomerase I inhibitor therapy and/or is known to be resistant to the topoisomerase I inhibitor therapy.

2. The method of claim 1, wherein the topoisomerase I inhibitor therapy is an irinotecan-based therapy.

3. The method of any one of claims 1-2, wherein the cancer is colorectal cancer, head and neck cancer, lung cancer, intrahepatic cholangiocarcinoma (iCCA), gastric cancer, urothelial cancer, endometrial cancer, cervical cancer, rhabdomyosarcoma, cholangiocarcinoma, glioblastoma, low-grade glioma, ovarian cancer, prostate adenocarcinoma, thyroid carcinoma, endometrial cancer, gallbladder cancer, breast cancer, or a combination thereof; optionally wherein the cancer is colorectal cancer; and further optionally the colorectal cancer is selected from the group consisting of colorectal adenoma, colon cancer, rectal cancer, colorectal carcinoma, colorectal adenocarcinoma, gastrointestinal carcinoid tumors, gastrointestinal stromal tumors (GISTs), primary colorectal lymphomas, colon leiomyosarcomas, rectal leiomyosarcomas, colon melanomas, rectal melanomas, colorectal squamous cell carcinoma, and a combination thereof.

4. The method of any one of claims 1-3, wherein the cancer is metastatic colorectal cancer (mCRC); optionally wherein the mCRC is associated with a RAS mutation; and further optionally the RAS mutation is an HRAS mutation, a KRAS mutation and/or an NRAS mutation.

5. The method of claim 4, wherein the RAS mutation is in codon 12, codon 13, codon 61 or a combination thereof, optionally wherein the RAS mutation is selected from the group consisting of KRASG12C, KRASG12D, KRASG12V, KRASG13C, KRASG13D, KRASQ61L, KRASQ61H, KRASQ61R, NRASQ61R, NRASQ61K, NRASQ61L, NRASQ61H, HRASG12V, HRASQ61R, HRASG12S and a combination thereof, and further optionally the RAS mutation is NRASQ61R, KRASG12C, KRASG12D, or KRASG12V.

6. The method of any one of claims 1-5, further comprising administrating an angiogenesis inhibitor to the subject; optionally wherein the angiogenesis inhibitor is selected from the group consisting of aflibercept, axitinib, bevacizumab, cabozantinib, 46envatinibb, pazopanib, ponatinib, ramucirumab, ranibizumab, regorafenib, sorafenib, sunitinib, vandetanib, and a combination thereof, and further optionally wherein the angiogenesis inhibitor is bevacizumab.

7. The method of any one of claims 1-6, further comprising administrating to the subject one or more additional therapeutic agents, and optionally wherein the one or more additional therapeutic agents comprise leucovorin, fluorouracil (5-FU) or a combination thereof.

8. The method of any one of claims 1-7, wherein onvansertib and the topoisomerase I inhibitor are co-administered simultaneously.

9. The method of any one of claims 1-7, wherein onvansertib and the topoisomerase I inhibitor are administered sequentially.

10. The method of any one of claims 6-9, wherein the angiogenesis inhibitor and/or the one or more additional therapeutic agents are co-administered simultaneously with onvansertib and/or the topoisomerase I inhibitor.

11. The method of any one of claims 6-9, wherein the angiogenesis inhibitor and/or the one or more additional therapeutic agents are administered sequentially with onvansertib and/or the topoisomerase I inhibitor.

12. The method of any one of claims 1-11, wherein onvansertib and the topoisomerase I inhibitor are administered through different routes.

13. The method of any one of claims 1-11, wherein onvansertib and the topoisomerase I inhibitor are administered through the same route.

14. The method of any one of claims 7-13, wherein the angiogenesis inhibitor and/or the one or more additional therapeutic agents are administered through different routes from onvansertib and/or the topoisomerase I inhibitor.

15. The method of any one of claims 7-13, wherein the angiogenesis inhibitor and/or the one or more additional therapeutic agents are administered through the same route as onvansertib and/or the topoisomerase I inhibitor.

16. The method of any one of claims 7-15, wherein one or more of onvansertib, the topoisomerase I inhibitor, the angiogenesis inhibitor, and the one or more additional therapeutic agents are administered orally, intraperitoneally, intravenously, subcutaneously, intramuscularly, intrathecally, or by inhalation.

17. The method of any one of claims 7-16, wherein one or more of onvansertib, the topoisomerase I inhibitor, the angiogenesis inhibitor, and the one or more additional therapeutic agents are administered in a cycle of at least about 7 days, a cycle of at least about 14 days, a cycle of at least about 21 days, or a cycle of at least about 28 days; optionally onvansertib, the topoisomerase I inhibitor, the angiogenesis inhibitor, and/or the one or more additional therapeutic agents are administered in a cycle of about 28 days; and further optionally wherein onvansertib is administered on at least four days in the cycle.

18. The method of claim 17, wherein the cycle is of 28 days and onvansertib is administered on days 1-5 and 15-19 in the cycle.

19. The method of any one of claims 17-18, wherein the topoisomerase I inhibitor, the angiogenesis inhibitor, and/or the one or more additional therapeutic agents are administered on at least one day in the cycle.

20. The method of any one of claims 17-19, wherein the cycle is of 28 days and the topoisomerase I inhibitor, the angiogenesis inhibitor, and/or the one or more additional therapeutic agents are administered on days 1 and 15 in the cycle.

21. The method of any one of claims 17-20, wherein the subject undergoes at least two cycles of administration.

22. The method of any one of claims 1-21, wherein onvansertib is administered at 8 mg/m²-90 mg/m², optionally wherein onvansertib is administered at 15 mg/m².

23. The method of any one of claims 1-22, wherein the topoisomerase I inhibitor is administered at 50-1000 mg/m², optionally at 180 mg/m².

24. The method of any one of claims 6-23, wherein the angiogenesis inhibitor is administered at 1-30 mg/kg, optionally wherein at 5 mg/kg.

25. The method of any one of claims 1-24, wherein a maximum concentration (Cₘₐₓ) of onvansertib in a blood of the subject is from about 100 nmol/L to about 1500 nmol/L after administration.

26. The method of any one of claims 1-25, wherein an area under curve (AUC) of a plot of a concentration of onvansertib in a blood of the subject over time is from about 1000 nmol/L·hour to about 400000 nmol/L·hour after administration.

27. The method of any one of claims 1-26, wherein a time (Tmax) to reach a maximum concentration of onvansertib in a blood of the subject is from about 1 hour to about 5 hours after administration.

28. The method of any one of claims 1-27, wherein an elimination half-life (T_{1/2}) of onvansertib in a blood of the subject is from about 10 hours to about 60 hours after administration.

29. The method of any one of claims 1-28, wherein the inhibition of progression of the cancer by the topoisomerase I inhibitor and onvansertib is greater than an added inhibition of progression of the cancer caused by the topoisomerase I inhibitor alone plus onvansertib alone.

30. The method of any one of claims 6-29, wherein the inhibition of progression of the cancer by the topoisomerase I inhibitor, the angiogenesis inhibitor and onvansertib is greater than an added inhibition of progression of the cancer caused by (1) the topoisomerase I inhibitor alone, the angiogenesis inhibitor alone and/or a combination of the topoisomerase I inhibitor and the angiogenesis inhibitor; plus (2) onvansertib alone.

31. The method of any one of claims 1-30, wherein the inhibition of progression of the cancer is measured by tumor growth inhibition (TGI) and wherein the TGI resulted by the treatment comprising the topoisomerase I inhibitor and onvansertib is at least 30% higher than the TGI of the topoisomerase I inhibitor alone.

32. The method of any one of claims 1-31, wherein a tumor volume after treatment with the topoisomerase I inhibitor and onvansertib decreases at least 50% relative to the tumor volume with no treatment.

33. The method of any one of claims 1-32, wherein the subject achieves a complete response.

34. The method of any one of claims 1-33, wherein the subject is a mammal, optionally wherein the mammal is human.

35. The method of any one of claims 1-34, wherein the subject has received at least one prior cancer treatment; optionally wherein the prior cancer treatment does not comprise the use of a topoisomerase I inhibitor, the angiogenesis inhibitor, onvansertib, or a combination thereof.

36. The method of any one of claims 1-35, wherein the subject was in remission for cancer, and optionally wherein the subject in remission for cancer was in complete remission (CR) or in partial remission (PR).

37. The method of any one of claims 1-36, wherein the subject has received a prior topoisomerase I inhibitor treatment, or wherein the subject did not respond to a treatment with the topoisomerase I inhibitor alone.

38. The method of any one of claims 1-37, further comprising determining responsiveness of the subject to onvansertib treatment.

39. A method of sensitizing cancer cells to a topoisomerase I inhibitor, the method comprising: contacting the cancer cells with a composition comprising onvansertib, thereby sensitizing the cancer cells to the topoisomerase I inhibitor; and optionally wherein contacting the cancer cells with the composition occurs in vitro, ex vivo, and/or in vivo.

40. The method of claim 39, wherein contacting the cancer cells with the composition is in a subject.

41. The method of any one of claims 39-40, comprising evaluating sensitization of the cancer cells to the topoisomerase I inhibitor after being contacted with onvansertib, and optionally wherein evaluating sensitization of the cancer cells to the topoisomerase I inhibitor comprises determining 1) colony forming capacity, 2) number of cancer cells in G2 and/or mitotic stages, 3) phosphorylation of NPM, 4) percentage of cancer cells containing phosphorylated histone H3 (pHH3), 5) percentage of cancer cells expressing cleaved caspase-3 and/or γ-H2AX, 6) expression of γ-H2AX, and/or 7) amount of cleaved caspase-3 and/or cleaved PARP.

42. The method of any one of claims 39-41, comprising contacting the cancer cells with the topoisomerase I inhibitor.

43. The method of any one of claims 39-42, wherein the cancer cells have at least one RAS mutation, and optionally wherein the RAS mutation is HRAS mutation, KRAS mutation and/or NRAS mutation.

44. The method of any one of claims 39-43, further comprising contacting the cancer cells with an angiogenesis inhibitor, and optionally bevacizumab.

45. The method of any one of claims 39-44, further comprising contacting the cancer cells with one or more additional therapeutic agents, optionally wherein the one or more additional therapeutic agents comprise leucovorin, fluorouracil (5-FU) or a combination thereof.

46. The method of any one of claims 1-45, wherein the topoisomerase I inhibitor is selected from the group consisting of irinotecan, topotecan, camptothecin, lamellarin D, 9-aminocamptothecin, SN-38, GG-211, DX-8951f, EGCG, genistein, quercetin, resveratrol and a combination thereof; optionally wherein the topoisomerase I inhibitor is irinotecan.

47. A method of treating cancer, the method comprising: administrating onvansertib and a chemotherapy comprising irinotecan to a subject with cancer, thereby inhibiting progression of the cancer, and wherein the subject has been treated with irinotecan and/or the subject is known to be resistant to an irinotecan treatment; and optionally wherein the chemotherapy comprises FOLFIRI and bevacizumab, and wherein FOLFIRI comprises irinotecan, leucovorin, and fluorouracil (5-FU).

48. A kit, comprising:
onvansertib; and
a manual providing instructions for administrating onvansertib with a topoisomerase I inhibitor to a subject for treating cancer, wherein the subject has been treated with irinotecan and/or the subject is known to be resistant to an irinotecan treatment,
optionally the topoisomerase I inhibitor is irinotecan.

49. The kit of claim 47, further comprising an angiogenesis inhibitor, and optionally the angiogenesis inhibitor is bevacizumab.

50. The kit of any one of claims 47-49, further comprising one or more additional therapeutic agents, optionally wherein the one or more additional therapeutic agents comprise leucovorin, fluorouracil (5-FU) or a combination thereof.
